(19) United States
(12) Patent Application Publication
(10) Pub. No.: US 2024/0225980 A9
(43) Pub. Date: Jul. 11, 2024

(54) METHODS AND FORMULATIONS FOR MITIGATING DAMAGING EFFECTS FROM EXPOSURE TO UV RADIATION

(71) Applicant: Innovation Hammer LLC, Powell, OH (US)

(72) Inventor: Arthur M. Nonomura, Litchfield Park, AZ (US)

(21) Appl. No.: 18/380,420

(22) Filed: Oct. 16, 2023

Related U.S. Application Data
(60) Provisional application No. 63/417,864, filed on Oct. 20, 2022.

(51) Int. Cl. A61K 8/49; A61Q 17/04

(52) U.S. Cl. CPC A61K 8/4973; A61Q 17/04

(57) ABSTRACT

Methods and formulations for the systemic protection of an animal from exposure to a photocarcinogen. The methods include orally administering to an animal a formulation comprising a therapeutically effective amount of one or more UV-absorbents and one or more stabilizers.

This application claims priority of U.S. Provisional Application Ser. No. 63/417,864 filed Oct. 20, 2022.

### BACKGROUND

The International Agency for Research on Cancer, World Health Organization (https://monographs.iarc.who.int/wp-content/uploads/2019/07/Classifications by_cancer_site) determined that solar radiation is a known carcinogen. Indeed, prolonged exposure to UV radiation induces genetic mutations in skin cells, the accepted cause of skin cancer, making UV radiation the definitive photocarcinogen.

Historically, prophylactic protection by topical application of sunscreens to exposed skin has been attempted by humans with limited success, but this method is inconvenient and, for the most part, inconsistent. Furthermore, topical sunscreens are prone to shedding and rinsing off, to the loss of protection and therefore require frequent topical re-applications. Indeed, humanity needs an effective system that protects globally from within, rather than by externally coating patches of skin for the prevention of injury from exposure to a photocarcinogen. Exposure to the UV spectrum is the root cause of photocarcinogenesis initiated by DNA-damage in skin and such a systemic protection would benefit humanity. Furthermore, exposure to UV radiation including from sunlight generates various superoxides, including peroxynitrates (ONOO⁻) and superoxide anions (O₂*⁻) that produce other radicals that are well-known in the field to cause light dermatoses and erythemas that produce outward signs of aging, and powerful protection from such radicals would benefit the health of skin.

Thus, there is a need for a formulation for systemic uptake that reduces the incidence of photocarcinogenesis via UV-absorbency and that proactively induces tissues to mitigate the deleterious effects of UV radiation, such as sunburn. Preferably the formulation will include one or more stabilizers of the active ingredient(s) while protecting them from degradation in the gut. In any bioactive substance, the control of its metabolic function is critical to its practical application and the embodiments herein disclose the discovery of regulation of UV-absorbency.

It therefore is an object of embodiments disclosed herein to provide formulations and methods useful for systemic protection against photocarcinogens. It is a further object of embodiments disclosed herein to provide formulations and methods for systemically protecting the skin of an animal, particularly a human, from certain deleterious effects from exposure to UV radiation, including one or more of skin wrinkling, skin creasing, skin sagging, skin thickening, fine lines, precancerous lesions, and/or skin cancer.

Another object of embodiments disclosed herein is a method of dosing for consistent systemic efficacy in animals, including a range of species from live mice to human beings. At the same time as protecting against photocarcinogens, the formulations disclosed herein in certain embodiments may provide mineral supplements for the intervention of certain dietary deficiencies. Moreover, it is an object of embodiments disclosed herein to apply methods to identify structures that have maximized UV-absorbency in Nature and, by further rational design, provide criteria for synthesis, stabilization of the formulations and methods for administration that achieve systemic efficacy in a live animal, including a human.

### SUMMARY

Problems of the prior art have been addressed by embodiments disclosed herein, which provide formulations and methods useful for systemic UV-absorbency and induction of bodily defenses against photocarcinogens, e.g., that provide for internally induced systemic protection and mitigation of and/or elimination of the deleterious effects of photocarcinogens. Furthermore, embodiments disclosed herein provide formulations for novel mineral supplements useful for the intervention of certain dietary deficiencies while affording UV protection. In addition, in certain embodiments, the formulations are stabilized to retain potency.

In certain embodiments, the methods and formulations disclosed herein are suitable for treating, mitigating or preventing one or more skin changes due to exposure to UV radiation, including improving or maintaining the appearance of skin. The one or more skin changes include changes to skin texture (e.g., uneven skin texture), skin wrinkling, skin aging, skin sagging, skin thickening, the appearance of fine lines, erythema, etc. In certain embodiments, the formulation is a cosmetic formulation.

In certain embodiments, disclosed are formulations comprising one or more UV-absorbents and one or more stabilizers.

In certain embodiments, the formulations consist essentially of one or more UV-absorbents and one or more stabilizers. As used herein, the phrase “consisting essentially of” or “consists essentially of” limits the scope of a claim to the specified materials or steps and those that do not materially affect the basic and novel characteristics of the claimed subject matter. The term permits the inclusion of elements or steps which do not materially affect the basic and novel characteristics of the apparatus, system or method under consideration. Accordingly, the expressions “consists essentially of” or “consisting essentially of” or the like mean that the recited embodiment, feature, component, step, etc. must be present and that other embodiments, features, components, steps, etc., may be present provided the presence thereof does not materially affect the performance, character or effect of the recited embodiment, feature, component, step, etc. The presence of an operation or step that has no material effect on the sample or product is permitted. For example, a formulation consisting essentially of a UV-absorbent (e.g., isolated 2-phenylchromenylium-acylₐglycoside_{g} where a represents an integer from 1-8, preferably from 1-5, and g represents an integer from 1 to 8) and a stabilizer may include other components, such as an excipient, buffer, etc., but not another active ingredient.

In certain embodiments, the UV-absorbent comprises one or more of the glycosylated-acylated-phenylchromenylium groups with the general structure, 2-phenylchromenylium-acylₐglycoside_{g} where a represents an integer from 1 to 8, preferably from 1 to 5, and g represents an integer from 1 to 8. In certain embodiments, the UV-absorbent comprises an isolated polyacyl 2-phenylchromenylium-acylₐglycoside_{g} where a represents an integer from 2 to 5 and g represents an integer from 1 to 8. In certain embodiments, the UV-absorbent is an isolated polyglycosyl 2-phenylchromenylium-acylₐglycoside_{g}, where a represents an integer from 1 to 5, and g represents an integer from 2 to 8.

In certain embodiments, the UV-absorbent comprises one or more isolated compounds, e.g., one or more 2-phenylchromenylium-acylₐglycosides_{g}, wherein a and g each independently represents an integer from 1-8, such as one or more compounds isolated from a purple carrot and/or black carrot, e.g., the atrorubens variety of Daucus carota subspecies saivus. In certain embodiments, the one or more isolated compounds is present in the formulation in a concentration higher than naturally occurs.

In certain embodiments, disclosed are UV-absorbents that comprise: a suitable (i) 3′,4′-dihydroxy-chromenylium-copigment; (ii) Acyl-copigment; (iii) Glycosyl-copigment; and (iv) metalⁿ⁺ copigment; and combinations of (i), (ii), (iii), (iv); where n in n+ is an integer from 2-4.

In certain embodiments, the formulations and methods are selected to achieve enhanced UV-absorbance in the postprandial state wherein the UV-absorbent comprises an isolated 3′,4′-dihydroxy-chromenylium metalⁿ⁺ copigment, referred to as the Meⁿ⁺ UV-absorbent. In certain embodiments, disclosed are formulations comprising one or more Meⁿ⁺ UV-absorbents comprised of one or more of the 2-phenyl-3′,4′-dihydroxy-chromenylium-acylₐglycoside_{g} Meⁿ⁺ where a represents an integer from 1-8, preferably from 1-5, and g represents an integer from 1 to 8. Said Meⁿ⁺ UV-absorbents may be comprised of selections from one or more of polyacyl 2-phenyl-3′,4′-dihydroxy-chromenylium-acylₐglycoside_{g} Meⁿ⁺ where a represents an integer from 2 to 5 and g represents an integer from 1 to 8; polyglycosyl 2-phenyl-3′,4′-dihydroxy-chromenylium-acylₐglycoside_{g} Meⁿ⁺ where a represents an integer from 1-8, preferably from 1-5, and g represents an integer from 2 to 8; and where n in n+ is an integer from 2-4.

In certain embodiments the formulation is suitable for oral administration, and the formulation is comprised of one or more stabilizer components that maintain postprandial stability (e.g., stability after consuming a meal) in the digestive tract of an animal to whom the formulation has been administered. When protected and stabilized, specific natural product UV-absorbents afford animals, including humans, powerful protection against damage from photocarcinogens and, further, afford animals, including humans, retention of a healthy skin appearance; e.g., skin free from or with mitigated wrinkles, thickening and/or sagging.

Another aspect of embodiments disclosed herein encompasses methods for modulating the generation of deleterious free radicals in an animal exposed to a photocarcinogen, comprising administering to said animal in need thereof a therapeutically effective amount of a formulation according to various embodiments described herein.

Accordingly, one aspect of embodiments disclosed herein encompasses formulations and methods for prophylaxis of sunburn and skin damage from photooxidation in an animal, including a human. In certain embodiments, the method for prophylaxis comprises administering to an animal in need thereof a therapeutically effective amount of a formulation according to the embodiments described herein. In certain embodiments, administration is carried out prior to exposure to UV radiation. In certain embodiments, administration is carried out prior to and/or during exposure to UV radiation. In certain embodiments, administration is carried out prior to and/or during and/or after exposure to UV radiation. In certain embodiments, the formulation comprises one or more Meⁿ⁺ UV-absorbents and one or more stabilizers.

Formulations disclosed herein may be orally administered, providing systemic protection in animals, including humans, and substantially reducing or eliminating deleterious effects of photocarcinogens, including the appearance of wrinkling effects of UV-induced sunburn (photowrinkling). In certain embodiments, the formulation is a drinkable potable beverage and/or a solid food that is orally administered with minimal or no systemic side effects (e.g., side effects such as deeper yellowness of urine or sublingual absorbance of color). In certain embodiments, the formulation is self-administered. In certain embodiments, the formulation is administered prior to anticipated UV exposure, especially prolonged (e.g., 5 minutes or more) UV exposure.

In certain embodiments, the formulation is administered to a human after UV exposure, especially after prolonged (e.g., 5 minutes or more) UV exposure. In certain embodiments, the formulations disclosed herein may be used together with the topical administration of conventional sunblock. The conventional sunblock may be applied to the skin prior to, during, and/or post-administration of the formulations disclosed herein.

In certain embodiments, formulations disclosed herein function as medicaments useful for ameliorating the appearance of aging, such as from the formation of skin wrinkles, fine lines, skin creasing, precancerous lesions, and/or skin thickening and sagging due to photodamage, e.g., due to exposure to UV radiation. Without suitable protection, as little as 3 minutes per day of exposure to UV radiation may increase the risk of skin cancer. In certain embodiments, the formulations are liquid or solid preparations, preferably aqueous solutions, such as elixirs, syrups, suspensions, emulsions, juices, teas, matcha, beverages, dairy, cocktails, and the like. In certain embodiments, the formulations are ingestible and/or potable, e.g., non-poisonous.

In certain embodiments, the formulations disclosed herein may protect against UV radiation from sources other than the sun, such as from tanning beds, sun lamps, mercury vapor lighting, lasers, halogen, fluorescent or incandescent lighting. In certain embodiments, the formulations disclosed herein may be useful for patients undergoing phototherapy.

In certain embodiments, disclosed is a method for the systemic protection of an animal from a photocarcinogen, comprising orally administering to said animal a therapeutically effective amount of a formulation comprising one or more Meⁿ⁺ UV-absorbents formulated with one or more stabilizers. In certain embodiments, disclosed are prophylactic formulations for systemic protection of an animal from a photocarcinogen. Such formulations may be administered to said animal in a therapeutically effective amount to systemically protect the animal from deleterious effects of exposure to a photocarcinogen. In certain embodiments, disclosed are prophylactic formulations for the systemic protection of an animal from a photocarcinogen that may be administered to said animal (including self-administration) in a therapeutically effective amount, wherein the formulations comprise one or more 2-(3,4-dihydroxyphenyl)chromenylium-3,5,7-triol 3-xylosyl(acylglucosyl)galactosides as Meⁿ⁺ UV-absorbents, where n in n+ represents an integer from 2-4; and one or more stabilizers selected from the group consisting of ascorbic acid, citric acid, malic acid, and salts thereof. In certain embodiments, the one or more stabilizers are present in amounts effective to achieve a formulation having a pH in the preferred range of 2-4.2, preferably 2-4.1.

In certain embodiments, the formulation comprises one or more Meⁿ⁺ UV-absorbent selected from the group consisting of polyglycosyl 2-phenyl-3′,4′-dihydroxy-chromenylium-glycosyl(acylaglycosylg)glycosides where a represents an integer from 1 to 8, and g represents an integer from 2 to 8, such as 2-(3,4-dihydroxyphenyl)chromenylium-3,5,7-triol 3-xylosyl(acylglucosyl)galactosides; 2-(3,4-dihydroxy-5-methoxyphenyl)chromenylium-3,5,7-triol 3-xylosyl(acylglucosyl)galactosides; 2-(3,4,5-trihydroxyphenyl)chromenylium-3,5,7-triol 3-xylosyl(acylglucosyl)galactosides; such as for example selected from the group consisting of 2-(3,4-dihydroxyphenyl)chromenylium-3,5,7-triol 3-xylosyl(feruloylglucosyl)galactosides, 2-(3,4-dihydroxyphenyl)chromenylium-3,5,7-triol 3-xylosyl(sinapoylglucosyl)galactosides, 2-(3,4-dihydroxyphenyl)chromenylium-3,5,7-triol 3-xylosyl(caffeoylglucosyl)galactosides, and 2-(3,4-dihydroxyphenyl)chromenylium-3,5,7-triol 3-xylosyl(coumaroylglucosyl)galactosides; and where n in n+ represents an integer from 2-4.

In certain embodiments, said formulation comprises one or more of said Meⁿ⁺ UV-absorbents comprising copigments that are extracted from a purple carrot and/or black carrot, preferably D. carota sativus var. atrorubens.

In certain embodiments, one or more Meⁿ⁺ UV-absorbents are selected from the group consisting of 2-phenylchromenylium-acylₐglycoside_{g}; 2-phenylchromenylium-3-xylosyl-(acylglucosyl)galactosides; 2-phenylchromenylium-3-xylosyl(sinapoylglucosyl)galactosides, 2-(3,4-dihydroxyphenyl)chromenylium-3,5,7-triol 3-(6-sinapoylgentiobioside), 2-(3,4-dihydroxyphenyl)chromenylium-3,5,7-triol 3-(maloylsuccinyl)glucosides, 2-(3,4-dihydroxyphenyl)chromenylium-3,5,7-triol 3-(malonylglucoside)s; 2-(3,4-dihydroxy-5-methoxyphenyl)chromenylium-3,5,7-triol 3-(p-coumaroyl-rutinoside)-5-glucoside; 3,4-dihydroxyphenyl)chromenylium-3,5,7-triol 3-(dimalonylglycosyl)glucosides; 2-(3,4-dihydroxyphenyl)chromenylium-3,5,7-triol 3-(coumaroylsophorosyl)-5-malonylglucosides; 2-(3,4-dihydroxyphenyl)chromenylium-3,5,7-triol 3-(feruloylsophorosyl)-5-malonylglucosides; 2-(3,4-dihydroxyphenyl)chromenylium-3,5,7-triol 3-(sinapoylcoumaroyl)sophorosyl-5-malonylglucosides, 2-(3,4-dihydroxyphenyl)chromenylium-3,5,7-triol 3-(feruloylglucosyl)-6-(glucosylferuloyl)(glucosylcoumaroyl)glucoside)-5-6-(malonylglucoside); 2-(3,4-dihydroxyphenyl)chromenylium-3,5,7-triol 3-(dimalonylglycosyl)glucosides, 2-(3,4-dihydroxyphenyl)chromenylium-3,5,7-triol 3-(maloylsuccinyl)glucosides, 2-(3,4-dihydroxyphenyl)chromenylium-3,5,7-triol 3-(coumaroylsophorosyl)-5-malonylglucosides, 2-(3,4-dihydroxyphenyl)chromenylium-3,5,7-triol 3-(feruloylsophorosyl)-5-malonylglucosides, 2-(3,4-dihydroxyphenyl)chromenylium-3,5,7-triol 3-(sinapoyl-coumaroyl)sophorosyl-5-malonylglucosides, 2-(3,4-dihydroxyphenyl)chromenylium-3,5,7-triol 3-(sinapoyl-feruloyl)-sophorosyl-5-malonylglucosides; alatanins; petanins; where n in n+ represents an integer from 2-4; a represents an integer from 1-8, preferably from 1-5, and g represents an integer from 1-8.

In certain embodiments, a prophylactic formulation for the systemic protection against damaging effects of UV radiation to the skin of an animal comprises one or more Meⁿ⁺ UV-absorbents comprising a 3′,4′-dihydroxy-chromenylium copigment in the presence of one or more stabilizers; and one or more optional Meⁿ⁺ copigments. In certain embodiments, a prophylactic formulation comprises a Meⁿ⁺ UV-absorbent comprising one or more Meⁿ⁺ copigments selected from Fe, Mg, Mn, V, and Zn, and combinations thereof, suitable for food use, that further may function as one or more mineral supplement, preferably as a treatment for mineral deficiencies. In certain embodiments, a prophylactic formulation for the systemic protection against damaging effects of UV radiation to the skin of an animal is selected from one or more Meⁿ⁺ UV-absorbents selected from the group consisting of 2-(3,4-dihydroxyphenyl)chromenylium-3,5,7-triol 3-xylosyl(acylaglucosylg)galactosides Meⁿ⁺; 2-(3,4-dihydroxy-5-methoxyphenyl)chromenylium-3,5,7-triol 3-xylosyl(acylaglucosylg)galactosides Meⁿ⁺; 2-(3,4,5-trihydroxyphenyl)chromenylium-3,5,7-triol 3-xylosyl(acylaglucosylg)galactosides Meⁿ⁺; 2-(3,4-dihydroxyphenyl)chromenylium-3,5,7-triol 3-(coumaroylsophoroside)-5-glucoside Meⁿ⁺; 2-(3,4-dihydroxyphenyl)chromenylium-3,5,7-triol 3-(coumaroyldiglucosyl)-5-glucoside Meⁿ⁺; 2-(3,4-dihydroxyphenyl)chromenylium-3,5,7-triol 3-(sinapoyldiglucosyl)-5-glucoside Meⁿ⁺; 2-(3,4-dihydroxyphenyl)chromenylium-3,5,7-triol 3-(dicaffeoylsophoroside)-5-glucoside Meⁿ⁺; 2-(3,4-dihydroxyphenyl)chromenylium-3,5,7-triol 3-(caffeoyl-6-feruloylsophoroside)-5-glucoside Meⁿ⁺; 2-(3,4-dihydroxyphenyl)chromenylium-3,5,7-triol 3-(p-hydroxybenzoyl-caffeoylsophoroside)-5-glucoside Meⁿ⁺; 2-(3,4-dihydroxyphenyl)chromenylium-3,5,7-triol 3-(coumaroylsophorosyl)-5-sinapoylglucoside Meⁿ⁺; 2-(3,4-dihydroxyphenyl)chromenylium-3,5,7-triol 3-xylosyl(sinapoylglucosyl)galactoside Meⁿ⁺; 2-(3,4-dihydroxyphenyl)chromenylium-3,5,7-triol 3-xylosyl(feruloylglucosyl)galactosides Meⁿ⁺; 2-(3,4-dihydroxyphenyl)chromenylium-3,5,7-triol 3-xylosyl(coumaroylglucosyl)galactosides Meⁿ⁺; 2-(3,4-dihydroxyphenyl)chromenylium-3,5,7-triol 3-xylosyl(caffeoylglucosyl)galactosides Meⁿ⁺; 2-(3,4-dihydroxy-5-methoxyphenyl)-chromenylium-3,5,7-triol 3-(p-coumaroylrutinoside)-5-glucoside Meⁿ⁺; 2-(3,4,5-trihydroxyphenyl)chromenylium-3,5,7-triol 3-(p-coumaroylrutinoside)-5-glucoside Meⁿ⁺; 2-(3,4-dihydroxyphenyl)chromenylium-3,5,7-triol 3-(maloylsuccinyl)glucosides Meⁿ⁺; 2-(3,4-dihydroxyphenyl)chromenylium-3,5,7-triol 3-malonylglucoside Meⁿ⁺; 2-(3,4-dihydroxyphenyl)chromenylium-3,5,7-triol 3-(dimalonylglycosyl)glucosides Meⁿ⁺; 2-(3,4-dihydroxyphenyl)chromenylium-3,5,7-triol 3-(coumaroylsophorosyl)-5-malonylglucosides Meⁿ⁺; 2-(3,4-dihydroxyphenyl)chromenylium-3,5,7-triol 3-(feruloylsophorosyl)-5-malonylglucosides Meⁿ⁺; 2-(3,4-dihydroxyphenyl)chromenylium-3,5,7-triol 3-(sinapoyl-coumaroyl)sophorosyl-5-malonylglucosides Meⁿ⁺; 2-(3,4-dihydroxyphenyl)chromenylium-3,5,7-triol 3-(sinapoyl-feruloyl)sophorosyl-5-malonylglucosides Meⁿ⁺; and 2-(3,4-dihydroxyphenyl)chromenylium-3,5,7-triol 3-(feruloylglucosyl)-6-(glucosylferuloyl)(glucosylcoumaroyl)glucoside)-5-6-(malonylglucoside) Meⁿ⁺; in the presence of one or more Meⁿ⁺ copigments selected from Fe, Mg, Mn, V, and Zn, where n in n+ represents an integer from 2-4; and where a represents an integer from 1-8, preferably from 1-5, and g represents an integer from 1-8. In certain embodiments, a prophylactic formulation comprises one or more Meⁿ⁺ UV-absorbents selected from 2-(3,4-dihydroxyphenyl)chromenylium-3,5,7-triol 3-xylosyl(acylglucosyl)galactosides Meⁿ⁺; 2-(3,4-dihydroxyphenyl)chromenylium-3,5,7-triol 3-xylosyl(feruloylglucosyl)galactosides Meⁿ⁺; 2-(3,4-dihydroxyphenyl)chromenylium-3,5,7-triol 3-xylosyl(sinapoylglucosyl)galactosides Meⁿ⁺; 2-(3,4-dihydroxyphenyl)chromenylium-3,5,7-triol 3-xylosyl(coumaroylglucosyl)galactosides Meⁿ⁺; 2-(3,4-dihydroxyphenyl)chromenylium-3,5,7-triol 3-xylosyl(caffeoylglucosyl)galactoside Meⁿ⁺; in the presence of one or more Meⁿ⁺ copigments selected from Fe, Mg, Mn, V, and Zn, where n in n+ represents an integer form 2-4; a represents an integer from 1-8, preferably from 1-5, and g represents an integer from 1 to 8. In certain embodiments, suitable Meⁿ⁺ UV-absorbents may be selected from the group consisting of Fe UV-absorbents as follow: 2-(3,4-dihydroxyphenyl)chromenylium-3,5,7-triol 3-(coumaroylsophoroside)-5-glucoside Fe; 2-(3,4-dihydroxyphenyl)chromenylium-3,5,7-triol 3-(coumaroyldiglucosyl)-5-glucoside Fe; 2-(3,4-dihydroxyphenyl)chromenylium-3,5,7-triol 3-(caffeoyl-6-feruloylsophoroside)-5-glucoside Fe; 2-(3,4-dihydroxyphenyl)chromenylium-3,5,7-triol 3-(p-hydroxybenzoyl-caffeoylsophoroside)-5-glucoside Fe; 2-(3,4-dihydroxyphenyl)chromenylium-3,5,7-triol 3-(coumaroylsophorosyl)-5-sinapoylglucoside Fe; 2-(3,4-dihydroxyphenyl)chromenylium-3,5,7-triol 3-xylosyl(feruloylglucosyl)galactosides Fe; 2-(3,4-dihydroxyphenyl)chromenylium-3,5,7-triol 3-xylosyl(coumaroylglucosyl)galactosides Fe; 2-(3,4,5-trihydroxyphenyl)chromenylium-3,5,7-triol 3-(p-coumaroylrutinoside)-5-glucoside Fe; 2-(3,4-dihydroxy-5-methoxyphenyl)chromenylium-3,5,7-triol 3-(p-coumaroylrutinoside)-5-glucoside Fe; 2-(3,4-dihydroxyphenyl)chromenylium-3,5,7-triol 3-xylosyl(caffeoylglucosyl)galactosides Fe; 2-(3,4-dihydroxyphenyl)chromenylium-3,5,7-triol 3-xylosyl(sinapoylglucosyl)galactoside Fe; 2-(3,4-dihydroxyphenyl)chromenylium-3,5,7-triol 3-(sinapoyldiglucosyl)-5-glucoside Fe; 2-(3,4-dihydroxyphenyl)chromenylium-3,5,7-triol 3-(dicaffeoylsophoroside)-5-glucoside Fe; 2-(3,4-dihydroxyphenyl)chromenylium-3,5,7-triol 3-(dimalonylglycosyl)glucosides Fe; 2-(3,4-dihydroxyphenyl)chromenylium-3,5,7-triol 3-(maloylsuccinyl)glucosides Fe; 2-(3,4-dihydroxyphenyl)chromenylium-3,5,7-triol 3-(coumaroylsophorosyl)-5-malonylglucosides Fe; 2-(3,4-dihydroxyphenyl)chromenylium-3,5,7-triol 3-(feruloylsophorosyl)-5-malonylglucosides Fe; 2-(3,4-dihydroxyphenyl)chromenylium-3,5,7-triol 3-(sinapoylcoumaroyl)sophorosyl-5-malonylglucosides Fe; 2-(3,4-dihydroxyphenyl)chromenylium-3,5,7-triol 3-(sinapoylferuloyl)sophorosyl-5-malonylglucosides Fe; 2-(3,4-dihydroxyphenyl)chromenylium-3,5,7-triol 3-malonylglucoside Fe; Meⁿ⁺ UV-absorbent in the presence of Fe copigment; and combinations thereof. In certain embodiments, suitable Meⁿ⁺ UV-absorbents may be selected from the group consisting of Zn UV-absorbents as follow: 2-(3,4-dihydroxyphenyl)chromenylium-3,5,7-triol 3-(coumaroylsophoroside)-5-glucoside Zn; 2-(3,4-dihydroxyphenyl)chromenylium-3,5,7-triol 3-(coumaroyldiglucosyl)-5-glucoside Zn; 2-(3,4-dihydroxyphenyl)chromenylium-3,5,7-triol 3-(caffeoyl-6-feruloylsophoroside)-5-glucoside Zn; 2-(3,4-dihydroxyphenyl)chromenylium-3,5,7-triol 3-(p-hydroxybenzoyl-caffeoylsophoroside)-5-glucoside Zn; 2-(3,4-dihydroxyphenyl)chromenylium-3,5,7-triol 3-(coumaroylsophorosyl)-5-sinapoylglucoside Zn; 2-(3,4-dihydroxyphenyl)chromenylium-3,5,7-triol 3-xylosyl(feruloylglucosyl)galactosides Zn; 2-(3,4-dihydroxyphenyl)chromenylium-3,5,7-triol 3-xylosyl(coumaroylglucosyl)galactosides Zn; 2-(3,4,5-trihydroxyphenyl)chromenylium-3,5,7-triol 3-(p-coumaroylrutinoside)-5-glucoside Zn; 2-(3,4-dihydroxy-5-methoxyphenyl)chromenylium-3,5,7-triol 3-(p-coumaroylrutinoside)-5-glucoside Zn; 2-(3,4-dihydroxyphenyl)chromenylium-3,5,7-triol 3-xylosyl(caffeoylglucosyl)galactosides Zn; 2-(3,4-dihydroxyphenyl)chromenylium-3,5,7-triol 3-xylosyl(sinapoylglucosyl)galactoside Zn; 2-(3,4-dihydroxyphenyl)chromenylium-3,5,7-triol 3-(sinapoyldiglucosyl)-5-glucoside Zn; 2-(3,4-dihydroxyphenyl)chromenylium-3,5,7-triol 3-(dicaffeoylsophoroside)-5-glucoside Zn; 2-(3,4-dihydroxyphenyl)chromenylium-3,5,7-triol 3-(dimalonylglycosyl)glucosides Zn; 2-(3,4-dihydroxyphenyl)chromenylium-3,5,7-triol 3-(maloylsuccinyl)glucosides Zn; 2-(3,4-dihydroxyphenyl)chromenylium-3,5,7-triol 3-(coumaroylsophorosyl)-5-malonylglucosides Zn; 2-(3,4-dihydroxyphenyl)chromenylium-3,5,7-triol 3-(feruloylsophorosyl)-5-malonylglucosides Zn; 2-(3,4-dihydroxyphenyl)chromenylium-3,5,7-triol 3-(sinapoylcoumaroyl)sophorosyl-5-malonylglucosides Zn; 2-(3,4-dihydroxyphenyl)chromenylium-3,5,7-triol 3-(sinapoylferuloyl)sophorosyl-5-malonylglucosides Zn; and 2-(3,4-dihydroxyphenyl)-chromenylium-3,5,7-triol 3-malonylglucoside Zn; in the presence of one or more Zn copigments; and where n in n+ represents an integer from 2-4; and combinations thereof. In certain embodiments, a prophylactic formulation comprises Meⁿ⁺ UV-absorbents selected from 2-(3,4-dihydroxyphenyl)chromenylium-3,5,7-triol 3-xylosyl(feruloylglucosyl)galactosides Fe and 2-(3,4-dihydroxyphenyl)chromenylium-3,5,7-triol 3-xylosyl(feruloylglucosyl)galactosides Zn; and where n in n+ represents an integer from 2-4.

In certain embodiments, a prophylactic formulation for the systemic protection against damaging effects of UV radiation to the skin of an animal comprises an effective amount of one or more Meⁿ⁺ UV-absorbents that may be administered in a dose of 0.5 to 12 grams Meⁿ⁺ UV-absorbent per day. In certain embodiments, the one or more Meⁿ⁺ UV-absorbents is present in the formulation in an amount of 0.1%-70% of the formulation; the amount of stabilizer is present in the formulation in an amount of 0.1-30% of the formulation in amounts effective to achieve a formulation having a preferable pH range of 2-4.2; and an amount of optional Meⁿ⁺ copigment of Meⁿ⁺ UV-absorbent is present in the formulation in an amount of 1 ppb-50% of the formulation; and where n in n+ represents an integer from 2-4.

In certain embodiments, the animal is a human. In certain embodiments the animal is a mammal. In certain embodiments the animal is a rodent, an ungulate, a horse, a pig, a cow or livestock.

In certain embodiments, the UV absorbent accumulates in the cells of an animal to whom it is administered.

In certain embodiments, oral administration of a therapeutically effective starter dose as taken 6-24 hours prior to exposure to a photocarcinogen may be in the range of 3 to 12 grams Meⁿ⁺ UV-absorbent; and from the day following said administration of a starter dose, an amount of one or more maintenance doses, in the range of 0.5 to 3 grams Meⁿ⁺ UV-absorbent, administered daily for the days, weeks, or months of exposures to a photocarcinogen; and where n in n+ represents an integer from 2-4.

In certain embodiments, oral administration of a therapeutically effective amount of one or more maintenance doses administered may be in the range of 0.5 to 3 grams Meⁿ⁺ UV-absorbent per day, as taken for 3 to 7 days or more prior to exposure to a photocarcinogen and onward daily maintenance doses administered in the range of 0.5 to 3 grams Meⁿ⁺ UV-absorbent per day; and where n in n+ represents an integer from 2-4.

In certain embodiments, the stabilizer is an acid and/or a salt thereof, preferably a Meⁿ⁺ organic acid, where n in n+ represents an integer from 2-4. In certain embodiments, the acid is a weak acid. In certain embodiments, the stabilizer is selected from the group of one or more of organic acids consisting of acetic acid, ascorbic acid, citric acid, fumaric acid, gluconic acid, lactic acid, and malic acid, and pharmaceutically acceptable salts thereof, the acid or salt preferably in amounts to achieve an acidic formulation in the preferred range of pH 2-4.2, preferably 2-4.1; and Meⁿ⁺ salts thereof, where n in n+ represents an integer from 2-4. In certain embodiments, the stabilizer is present in an amount in the range of 0.1-30%, that is effective at maintaining the pH of the formulation from about 1-4.5, preferably at about the range of pH 2 to 4.2, and more preferably in the range of pH 2 to 4.1.

In certain embodiments, the source of the photocarcinogen is one or more of sunlight, UVA and UVB.

In certain embodiments, the prophylactic formulation is contained in a housing impervious to light, including cans (e.g., aluminum cans), amber or brown bottles (e.g., glass bottles, plastic bottles made of one or more polymers, such as polyethylene terephthalate), or any other UV light impervious container.

In certain embodiments, the components of the formulation are selected for stability and uptake by an animal.

In certain embodiments, the formulation or parts thereof, are adjuvants, administered in conjunction with a vaccine, a mineral supplement, and/or other medical therapies.

Accordingly, in certain embodiments disclosed is a method for the systemic protection of an animal from exposure to a photocarcinogen, comprising orally administering to the animal a therapeutically effective amount of a formulation comprising one or more metalⁿ⁺ UV-absorbents comprising one or more 2-phenylchromenylium-acylₐglycoside_{g} metalⁿ⁺ and one or more stabilizers present in the formulation in an amount effective to achieve a pH of from 1-4.5 of the formulation; and wherein a and g each independently represents an integer from 1-8 and n in n+ represents an integer from 2-4. In certain embodiments, a represents an integer from 1-5. In certain embodiments, the animal is a human.

In certain embodiments, the oral administration comprises a starter dose taken 6-24 hours prior to said exposure to a photocarcinogen, in the range of 3 to 12 grams metalⁿ⁺ UV-absorbent; and a maintenance dose taken the day following said administration of the starter dose, the maintenance dose being in the range 0.5 to 3 grams metalⁿ⁺ UV-absorbent; and where n in n+ represents an integer from 2-4. In certain embodiments, the therapeutically effective amount of the formulation comprises 0.5 to 3 grams of the one or more metalⁿ⁺ UV-absorbents administered per day, taken for 3 to 7 days prior to exposure to a photocarcinogen, followed by 0.5 to 3 grams metalⁿ⁺ UV-absorbent administered per day for the duration of the exposure of the animal to a photocarcinogen; and where n in n+ represents an integer from 2-4.

In certain embodiments, the metalⁿ⁺ UV-absorbent is selected from the group consisting of: (i) 3′,4′-dihydroxy-chromenylium-copigment selected from 2-(3,4-dihydroxyphenyl)chromenylium-3,5,7-triol, 2-(3,4-dihydroxy-5-methoxyphenyl)chromenylium-3,5,7-triol, and (2-(3,4,5-trihydroxyphenyl)chromenylium-3,5,7-triol; (ii) Acyl-copigment selected from the group consisting of aliphatic acids, acetic, malic, malonic, oxalic, succinic, tartaric acids; aromatic acids, caffeic, coumaric, ferulic, gallic, hydroxybenzoic, and sinapic acids; acyl, oligoacyl, polyacyl, thereof, (iii) Glycosyl-copigment selected from the group consisting of hexoses, glucose, rhamnose, galactose, pentoses, xylose, arabinose, rutinose, sophorose, samubiose; glycosides, oligoglycosyl, and polyglycosyl derivatives thereof; and (iv) Metalⁿ⁺ copigment selected from the group consisting of Fe, Mg, Mn, V, Zn and combinations thereof; and combinations of (i), (ii), (iii) and/or (iv). In certain embodiments, the metalⁿ⁺ UV-absorbent is selected from the group consisting of 2-(3,4-dihydroxyphenyl)chromenylium-3,5,7-triol 3-xylosyl(acylglucosyl)galactosides metalⁿ⁺; 2-(3,4-dihydroxyphenyl)chromenylium-3,5,7-triol 3-xylosyl(feruloylglucosyl)galactosides metalⁿ⁺; 2-(3,4-dihydroxyphenyl)chromenylium-3,5,7-triol 3-xylosyl(sinapoylglucosyl)galactosides metalⁿ⁺; 2-(3,4-dihydroxyphenyl)chromenylium-3,5,7-triol 3-xylosyl(coumaroylglucosyl)galactosides metalⁿ⁺; 2-(3,4-dihydroxyphenyl)chromenylium-3,5,7-triol 3-xylosyl(caffeoylglucosyl)galactoside metalⁿ⁺; and combinations thereof, in the presence of a metalⁿ⁺⁻ selected from the group consisting of Fe, Mg, Mn, V, and Zn and combinations thereof; and where n in n+ represents an integer from 2-4.

In certain embodiments, a source of the one or more metalⁿ⁺ UV-absorbents comprises an extract from purple carrot, Daucus carota sativus var. atrorubens; in the presence of a metalⁿ⁺ copigment selected from the group consisting of Fe, Mg, Mn, V, and Zn and combinations thereof.

In certain embodiments, the metalⁿ⁺ UV-absorbent is selected from the group consisting of 2-(3,4-dihydroxyphenyl)chromenylium-3,5,7-triol 3-acylₐglycoside_{g} metalⁿ⁺; 2-(3,4-dihydroxy-5-methoxyphenyl)chromenylium-3,5,7-triol 3-acylₐglycoside_{g} metalⁿ⁺; 2-(3,4,5-trihydroxyphenyl)chromenylium-3,5,7-triol 3-acylₐglycoside_{g} metalⁿ⁺; 2-(3,4-dihydroxyphenyl)chromenylium-3,5,7-triol 3-(coumaroylsophoroside)-5-glucoside metalⁿ⁺; 2-(3,4-dihydroxyphenyl)chromenylium-3,5,7-triol 3-(coumaroyldiglucosyl)-5-glucoside metalⁿ⁺; 2-(3,4-dihydroxyphenyl)chromenylium-3,5,7-triol 3-(sinapoyldiglucosyl)-5-glucoside metalⁿ⁺; 2-(3,4-dihydroxyphenyl)chromenylium-3,5,7-triol 3-(dicaffeoylsophoroside)-5-glucoside metalⁿ⁺; 2-(3,4-dihydroxyphenyl)chromenylium-3,5,7-triol 3-(caffeoyl-6-feruloylsophoroside)-5-glucoside metalⁿ⁺; 2-(3,4-dihydroxyphenyl)chromenylium-3,5,7-triol 3-(p-hydroxybenzoyl-caffeoylsophoroside)-5-glucoside metalⁿ⁺; 2-(3,4-dihydroxyphenyl)chromenylium-3,5,7-triol 3-(coumaroylsophorosyl)-5-sinapoylglucoside metalⁿ⁺; 2-(3,4-dihydroxyphenyl)chromenylium-3,5,7-triol 3-xylosyl(sinapoylglucosyl)galactoside metalⁿ⁺; 2-(3,4-dihydroxyphenyl)chromenylium-3,5,7-triol 3-xylosyl(feruloylglucosyl)galactosides metalⁿ⁺; 2-(3,4-dihydroxyphenyl)chromenylium-3,5,7-triol 3-xylosyl(coumaroylglucosyl)galactosides metalⁿ⁺; 2-(3,4-dihydroxyphenyl)chromenylium-3,5,7-triol 3-xylosyl(caffeoylglucosyl)galactosides metalⁿ⁺; 2-(3,4-dihydroxy-5-methoxyphenyl)chromenylium-3,5,7-triol 3-(p-coumaroylrutinoside)-5-glucoside metalⁿ⁺; 2-(3,4,5-trihydroxyphenyl)chromenylium-3,5,7-triol 3-(p-coumaroylrutinoside)-5-glucoside metalⁿ⁺; 2-(3,4-dihydroxyphenyl)chromenylium-3,5,7-triol 3-(maloylsuccinyl)glucosides metalⁿ⁺; 2-(3,4-dihydroxyphenyl)chromenylium-3,5,7-triol 3-malonylglucoside metalⁿ⁺; 2-(3,4-dihydroxyphenyl)chromenylium-3,5,7-triol 3-(dimalonylglycosyl)glucosides metalⁿ⁺; 2-(3,4-dihydroxyphenyl)chromenylium-3,5,7-triol 3-(coumaroylsophorosyl)-5-malonylglucosides metalⁿ⁺; 2-(3,4-dihydroxyphenyl)chromenylium-3,5,7-triol 3-(feruloylsophorosyl)-5-malonylglucosides metalⁿ⁺; 2-(3,4-dihydroxyphenyl)chromenylium-3,5,7-triol 3-(sinapoyl-coumaroyl)sophorosyl-5-malonylglucosides metalⁿ⁺; 2-(3,4-dihydroxyphenyl)chromenylium-3,5,7-triol 3-(sinapoyl-feruloyl)sophorosyl-5-malonylglucosides metalⁿ⁺; 2-(3,4-dihydroxyphenyl)chromenylium-3,5,7-triol 3-(feruloylglucosyl)-6-(glucosylferuloyl)(glucosylcoumaroyl)glucoside)-5-6-(malonylglucoside) metalⁿ⁺, and combinations thereof; in the presence of one or more metalⁿ⁺ copigments selected from the group consisting of Fe, Mg, Mn, V and Zn, where n in n+ represents an integer from 2-4; and where a represents an integer from 1-5, and g represents an integer from 1 to 8.

In certain embodiments, the one or more stabilizers is selected from the group consisting of acetic acid, ascorbic acid, carbonic acid, citric acid, fumaric acid, gluconic acid, glutaric acid, lactic acid, malic acid, phosphoric acid, succinic acid, tartaric acid and metalⁿ⁺ salts thereof; and where n in n+ represents an integer from 2-4.

In certain embodiments, the photocarcinogen is one or more of sunlight, UVA and UVB.

In certain embodiments, the administration improves the appearance of skin of said animal. In certain embodiments, the improvement of the appearance of skin comprises the mitigation of at least one of skin wrinkling, skin sagging, skin thickening, and fine lines. In certain embodiments, the administration mitigates or prevents photowrinkling of skin of said animal.

In certain embodiments, disclosed is a formulation for the systemic protection against damaging effects of UV radiation to the skin of an animal, comprising one or more metalⁿ⁺ UV-absorbents comprising a 2-phenylchromenylium-acylₐglycoside_{g} metalⁿ⁺ copigment in an amount of 0.1%-70% of the formulation, wherein a and g each independently represent an integer from 1-8 and n in n+ represents an integer from 2-4; and one or more stabilizers in an amount of 0.1-30% of the formulation so as to achieve a pH of said formulation in the range of 1-4.5. In certain embodiments, the metalⁿ⁺ UV-absorbent in the formulation is selected from the group consisting of: (i) a 3′,4′-dihydroxy-chromenylium copigment selected from 2-(3,4-dihydroxyphenyl)chromenylium-3,5,7-triol and 2-(3,4-dihydroxy-5-methoxyphenyl)chromenylium-3,5,7-triol; 2-(3,4,5-trihydroxyphenyl)chromenylium-3,5,7-triol); (ii) Acyl-copigment comprising one or more of aliphatic acids, acetic, malic, malonic, oxalic, succinic, tartaric acids; aromatic acids, caffeic, coumaric, ferulic, gallic, hydroxybenzoic, and sinapic acids; and acyl, oligoacyl and polyacyl derivatives thereof; (iii) Glycosyl-copigment comprising one or more of hexoses, glucose, rhamnose, galactose, pentoses, xylose, arabinose, rutinose, sophorose, samubiose; glycoside, oligoglycosyl and polyglycosyl derivatives thereof, (iv) Metalⁿ⁺ copigment comprising one or more of Fe, Mg, Mn, V, and Zn; combinations of (i), (ii), (iii) and (iv); where n in n+ represents an integer from 2-4; and derivatives thereof.

In certain embodiments, the metalⁿ⁺ UV-absorbent in the formulation comprises a 3′,4′-dihydroxy-chromenylium copigment selected from the group consisting of 2-phenyl-3′,4′-dihydroxy-chromenylium-acylₐglycoside_{g} metalⁿ⁺; 2-(3,4-dihydroxyphenyl)chromenylium-3,5,7-triol 3-acylₐglycoside_{g} metalⁿ⁺, 2-(3,4,5-trihydroxyphenyl)chromenylium-3,5,7-triol 3-acylₐglycoside_{g} metalⁿ⁺; 2-(3,4-dihydroxy-5-methoxyphenyl)chromenylium-3,5,7-triol 3-acylₐglycoside_{g} metalⁿ⁺; 2-(3,4-dihydroxyphenyl)chromenylium-3,5,7-triol 3-xylosyl(sinapoylglucosyl)galactoside metalⁿ⁺, 2-(3,4-dihydroxyphenyl)chromenylium-3,5,7-triol 3-xylosyl(feruloylglucosyl)galactosides metalⁿ⁺, 2-(3,4-dihydroxyphenyl)chromenylium-3,5,7-triol 3-xylosyl(coumaroylglucosyl)galactosides metalⁿ⁺, 2-(3,4-dihydroxyphenyl)chromenylium-3,5,7-triol 3-xylosyl(caffeoylglucosyl)galactosides metalⁿ⁺; 2-(3,4-dihydroxyphenyl)chromenylium-3,5,7-triol 3-(coumaroylsophoroside)-5-glucoside metalⁿ⁺; 2-(3,4-dihydroxyphenyl)chromenylium-3,5,7-triol 3-(coumaroyldiglucosyl)-5-glucoside metalⁿ⁺; 2-(3,4-dihydroxyphenyl)chromenylium-3,5,7-triol 3-(sinapoyldiglucosyl)-5-glucoside metalⁿ⁺; 2-(3,4-dihydroxyphenyl)chromenylium-3,5,7-triol 3-(dicaffeoylsophoroside)-5-glucoside metalⁿ⁺; 2-(3,4-dihydroxyphenyl)chromenylium-3,5,7-triol 3-(caffeoyl-6-feruloylsophoroside)-5-glucoside metalⁿ⁺; 2-(3,4-dihydroxyphenyl)chromenylium-3,5,7-triol 3-(p-hydroxybenzoyl-caffeoylsophoroside)-5-glucoside metalⁿ⁺; 2-(3,4-dihydroxyphenyl)chromenylium-3,5,7-triol 3-(coumaroylsophorosyl)-5-sinapoylglucoside metalⁿ⁺; 2-(3,4,5-trihydroxyphenyl)chromenylium-3,5,7-triol 3-(p-coumaroylrutinoside)-5-glucoside metalⁿ⁺; 2-(3,4-dihydroxy-5-methoxyphenyl)chromenylium-3,5,7-triol 3-(p-coumaroylrutinoside)-5-glucoside metalⁿ⁺; 2-(3,4-dihydroxyphenyl)chromenylium-3,5,7-triol 3-malonylglucoside metalⁿ⁺; 2-(3,4-dihydroxyphenyl)chromenylium-3,5,7-triol 3-(dimalonylglycosyl)glucosides metalⁿ⁺; 2-(3,4-dihydroxyphenyl)chromenylium-3,5,7-triol 3-(maloylsuccinyl)glucosides metalⁿ⁺; 2-(3,4-dihydroxyphenyl)chromenylium-3,5,7-triol 3-(coumaroylsophorosyl)-5-malonylglucosides metalⁿ⁺; 2-(3,4-dihydroxyphenyl)chromenylium-3,5,7-triol 3-(feruloylsophorosyl)-5-malonylglucosides metalⁿ⁺; 2-(3,4-dihydroxyphenyl)chromenylium-3,5,7-triol 3-(sinapoyl-coumaroyl)sophorosyl-5-malonylglucosides metalⁿ⁺; 2-(3,4-dihydroxyphenyl)chromenylium-3,5,7-triol 3-(sinapoyl-feruloyl)sophorosyl-5-malonylglucosides metalⁿ⁺; and 2-(3,4-dihydroxyphenyl)chromenylium-3,5,7-triol 3-(feruloylglucosyl)-6-(glucosylferuloyl)(glucosylcoumaroyl)glucoside)-5-6-(malonylglucoside) metalⁿ⁺; in the presence of metalⁿ⁺ copigments selected from the group consisting of Fe, Mg, Mn, Zn and combinations thereof, where n in n+ represents an integer from 2-4; a represents an integer from 1-5, g represents an integer from 1-8, and n in n+ represents an integer from 2-4.

In certain embodiments, the comprises one or more inducers comprising a riboflavin Na phosphate; the inducer further comprising one or more ammonium compounds selected from the group consisting of ammonium sulfate, NH₄HCO₃, and C₄₂H₆₅NO₁₆; the inducer being present in the formulation in the amount from 1 to 100 ppm.

In certain embodiments, the one or more metalⁿ⁺ UV-absorbents in the formulation is selected from the group consisting of 2-(3,4-dihydroxyphenyl)chromenylium-3,5,7-triol 3-xylosyl(acylglucosyl)galactosides Fe; 2-(3,4-dihydroxyphenyl)chromenylium-3,5,7-triol 3-xylosyl(feruloylglucosyl)galactosides Fe; 2-(3,4-dihydroxyphenyl)chromenylium-3,5,7-triol 3-xylosyl(sinapoylglucosyl)galactosides Fe; 2-(3,4-dihydroxyphenyl)chromenylium-3,5,7-triol 3-xylosyl(coumaroylglucosyl)galactosides Fe; 2-(3,4-dihydroxyphenyl)chromenylium-3,5,7-triol 3-xylosyl(caffeoylglucosyl)galactoside Fe; 2-(3,4-dihydroxyphenyl)chromenylium-3,5,7-triol 3-xylosyl(acylglucosyl)galactosides Zn; 2-(3,4-dihydroxyphenyl)chromenylium-3,5,7-triol 3-xylosyl(feruloylglucosyl)galactosides Zn; 2-(3,4-dihydroxyphenyl)chromenylium-3,5,7-triol 3-xylosyl(sinapoylglucosyl)galactosides Zn; 2-(3,4-dihydroxyphenyl)chromenylium-3,5,7-triol 3-xylosyl(coumaroylglucosyl)galactosides Zn; and 2-(3,4-dihydroxyphenyl)chromenylium-3,5,7-triol 3-xylosyl(caffeoylglucosyl)galactoside Zn; in the presence of one or more metalⁿ⁺ copigments selected from Fe and Zn, wherein n in n+ represents an integer from 2-4; and mineral supplements thereof.

In certain embodiments, the is contained in a housing impervious to light.

In certain embodiments, the one or more metalⁿ⁺ UV-absorbents in the formulation is selected from the group consisting of 2-(3,4-dihydroxyphenyl)chromenylium-3,5,7-triol 3-acylₐglycoside_{g} metalⁿ⁺ in an amount of 0.5-12 grams dry weight; and wherein said formulation comprises ascorbic acid in the amount of 10-1000 mg dry weight; flavins in the amount of 1-100 ppm dry weight; ammonium bicarbonate in the amount of 1-100 ppm dry weight; zinc ascorbate in the amount of 0.01-50 mg dry weight; and ferrous fumarate in the amount of 1 mcg-50 mg dry weight; wherein said formulation is for a human dose; where a represents an integer from 1-5, g represents an integer from 1 to 8, and n in n+ represents an integer from 2-4.

In certain embodiments, disclosed is a formulation for the systemic protection against damaging effects of UV radiation to skin of an animal, comprising one or more metalⁿ⁺ UV-absorbents selected from the group consisting of 2-phenylchromenylium-acylₐglycoside_{g} metalⁿ⁺; 2-(3,4-dihydroxyphenyl)chromenylium-3,5,7-triol 3-acylₐglycoside_{g} metalⁿ⁺; 2-(3,4-dihydroxyphenyl)chromenylium-3,5,7-triol 3-xylosyl(sinapoylglucosyl)galactoside metalⁿ⁺, 2-(3,4-dihydroxyphenyl)chromenylium-3,5,7-triol 3-xylosyl(feruloylglucosyl)galactosides metalⁿ⁺, 2-(3,4-dihydroxyphenyl)chromenylium-3,5,7-triol 3-xylosyl(coumaroylglucosyl)galactosides metalⁿ⁺, 2-(3,4-dihydroxyphenyl)chromenylium-3,5,7-triol 3-xylosyl(caffeoylglucosyl)galactosides metalⁿ⁺; 2-(3,4,5-trihydroxyphenyl)chromenylium-3,5,7-triol 3-acylₐglycoside_{g} metalⁿ⁺; 2-(3,4-dihydroxy-5-methoxyphenyl)chromenylium-3,5,7-triol 3-acylₐglycoside_{g} metalⁿ⁺; 2-(3,4-dihydroxyphenyl)chromenylium-3,5,7-triol 3-(coumaroylsophoroside)-5-glucoside metalⁿ⁺; 2-(3,4-dihydroxyphenyl)chromenylium-3,5,7-triol 3-(coumaroyldiglucosyl)-5-glucoside metalⁿ⁺; 2-(3,4-dihydroxyphenyl)chromenylium-3,5,7-triol 3-(sinapoyldiglucosyl)-5-glucoside metalⁿ⁺; 2-(3,4-dihydroxyphenyl)chromenylium-3,5,7-triol 3-(dicaffeoylsophoroside)-5-glucoside metalⁿ⁺; 2-(3,4-dihydroxyphenyl)chromenylium-3,5,7-triol 3-(caffeoyl-6-feruloylsophoroside)-5-glucoside metalⁿ⁺; 2-(3,4-dihydroxyphenyl)chromenylium-3,5,7-triol 3-(p-hydroxybenzoyl-caffeoylsophoroside)-5-glucoside metalⁿ⁺; 2-(3,4-dihydroxyphenyl)chromenylium-3,5,7-triol 3-(coumaroylsophorosyl)-5-sinapoylglucoside metalⁿ⁺; 2-(3,4,5-trihydroxyphenyl)chromenylium-3,5,7-triol 3-(p-coumaroylrutinoside)-5-glucoside metalⁿ⁺; 2-(3,4-dihydroxy-5-methoxyphenyl)chromenylium-3,5,7-triol 3-(p-coumaroylrutinoside)-5-glucoside metalⁿ⁺; 2-(3,4-dihydroxyphenyl)chromenylium-3,5,7-triol 3-malonylglucoside metalⁿ⁺; 2-(3,4-dihydroxyphenyl)chromenylium-3,5,7-triol 3-(dimalonylglycosyl)glucosides metalⁿ⁺; 2-(3,4-dihydroxyphenyl)chromenylium-3,5,7-triol 3-(maloylsuccinyl)glucosides metalⁿ⁺; 2-(3,4-dihydroxyphenyl)chromenylium-3,5,7-triol 3-(coumaroylsophorosyl)-5-malonylglucosides metalⁿ⁺; 2-(3,4-dihydroxyphenyl)chromenylium-3,5,7-triol 3-(feruloylsophorosyl)-5-malonylglucosides metal*; 2-(3,4-dihydroxyphenyl)chromenylium-3,5,7-triol 3-(sinapoyl-coumaroyl)sophorosyl-5-malonylglucosides metalⁿ⁺; and 2-(3,4-dihydroxyphenyl)chromenylium-3,5,7-triol 3-(sinapoyl-feruloyl)sophorosyl-5-malonylglucosides metalⁿ⁺; and 2-(3,4-dihydroxyphenyl)chromenylium-3,5,7-triol 3-(feruloylglucosyl)-6-(glucosylferuloylglucosylcoumaroyl)glucoside)-5-6-(malonylglucoside) metalⁿ⁺; in the presence of one or more metalⁿ⁺ copigments selected from the group consisting of Fe, Mg, Mn, V and Zn, where n in n+ represents an integer from 2-4; a represents an integer from 1-5 and g represents an integer from 1-8; and combinations thereof.

### BRIEF DESCRIPTION OF THE DRAWINGS

FIG. 1 is a diagram of the reactions and transformations of the superoxide anion Oz-;

FIG. 2 is a graph showing significantly reduced formation of sunburn cells in murine skin following treatment with HB7 (Treatment, right) as compared to Controls (left);

FIG. 3 is a graph showing significantly reduced epidermal thickness in murine skin following treatment with HB7 (Treatment, right) as compared to Controls (left);

FIG. 4 is a graph showing significantly reduced cyclobutane pyrimidine dimers (CPD+) formation and oxidative stress following treatment with HB7 (Treatment, right) as compared to Controls (left);

FIG. 5 is a graph showing significantly reduced 8-oxo-2′-deoxyguanosine (8-oxo-dG) oxidative stress formation and oxidative stress following treatment with HB7 (Treatment, right) as compared to Controls (left); and

FIG. 6 is a photograph showing a shift to absorbance of sunlight irradiation after formation of the CyaCFS Fe coordination complex (CyaCFS+iron(II), right) that is darker, as compared to the CyaCFS Control solution (left) that is lighter, exhibiting less absorbance.

### DETAILED DESCRIPTION

A more complete understanding of the components and methods disclosed herein can be obtained by reference to the accompanying drawings. Although specific terms are used in the following description for the sake of clarity, these terms are intended to refer only to the particular structure of the embodiments selected for illustration in the drawing and are not intended to define or limit the scope of the disclosure. In the drawing and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

Unless defined otherwise, all technical and scientific terms used herein have the same meaning as commonly understood by one of ordinary skill in the art. All publications, patents and other references herein mentioned are incorporated by reference and applicable chemicals are given according to scientific nomenclature known to the field. As used herein, “Al” refers to aluminum; “Cu” refers to copper; “Fe” refers to iron(II)(III); “FeSO₄·7H₂O” refers to iron(II) sulfate heptahydrate; “H₂O₂” refers to hydrogen peroxide; “Mg” refers to magnesium; “Metalⁿ⁺” and “Meⁿ⁺” refer to n*-valent metal copigments of UV-absorbents, where n in n+ represents an integer from 2-4; “Metal²⁺” and “Me²⁺” refer to divalent metalⁿ⁺ copigments of a UV-absorbent; “Mn” refers to manganese; “Mo” refers to molybdenum; “N” refers to nitrogen; “Na” refers to sodium; “NH₄HCO₃” refers to ammonium bicarbonates; “Ni” refers to nickel; “C₄₂H₆₅NO₁₆” refers to ammonium glycyrrhizates; “O” refers to oxygen; “O₂^{*-}” refers to superoxide anions; “ONOO” refers to peroxynitrates; “V” refers to vanadium; and “Zn” refers to zinc or divalent zinc(II).

As used herein, a percentage, %, or concentration refers to a percentage or concentration by weight; “ppt” refers to parts per thousand by weight and is equal to 0.1% or 1 gram/liter (1 gm/L); “ppm” refers to parts per million by weight and is equivalent to 1 milligram/liter (1 mg/L); “ppb” refers to parts per billion by weight and is equivalent to 1 microgram/liter (1 mcg/L); and all ranges are inclusive of the endpoints.

As used herein, the term “therapeutically effective amount” or the like includes the amount of one or more formulations disclosed herein that when administered to an animal for treating, mitigating or preventing a state, disease, disorder or condition associated with or caused by photooxidation is sufficient to effect such treatment, mitigation or prevention. A “therapeutically effective amount” or the like includes an amount sufficient to cause a cosmetic improvement to the skin of an animal when one or more formulations disclosed herein are administered to such animal. The “therapeutically effective amount” may vary depending on the particular state, disease, disorder or condition being treated and its severity and the age, weight, height, gender, physical condition, and responsiveness of the animal to be treated. Thus, one or more of these parameters can be used to select and adjust the therapeutically effective amount. Also, the amount can be determined using pharmacologic methods known in the art, such as dose response curves.

As used herein, “treatment or treating” refers to eliminating, improving, mitigating, preventing, or otherwise reducing the presence or signs of an unhealthy condition. “Treatment” or “treating” of a state, disorder or condition and may include: (1) preventing or delaying the appearance or development of the clinical symptoms of the state, disease, disorder or condition associated with or caused by photooxidation or photocarcinogenesis of an animal that may be afflicted with or predisposed to the state, disease, disorder or condition, but does not yet experience or display clinical or subclinical symptoms of the state, disease, disorder or condition, (2) inhibiting the state, disease, disorder or condition associated with or caused by photooxidation or photocarcinogenesis, e.g., arresting or reducing the development of the state, disease, disorder or condition associated with or caused by photooxidation or at least one clinical or subclinical symptom thereof, (3) relieving or ameliorating the state, disease, disorder or condition associated with or caused by photooxidation or photocarcinogenesis, e.g., causing regression or amelioration of the state, disease, disorder or condition associated with or caused by photooxidation or photocarcinogenesis or at least one of its clinical or subclinical symptoms, or (4) relieving or ameliorating the state, disease, disorder or condition associated with or caused by a deficiency of essential mineral nutrition, e.g., causing regression or amelioration of the state, disease, disorder or condition associated with or caused by mineral deficiency or at least one of its clinical or subclinical symptoms.

As used herein, “UV-absorbent” refers to water-soluble and/or alcohol-soluble that absorb in the range of the UV-spectrum. In certain embodiments, the UV-absorbents are formulated in the broad range of 0.1 to 70% of a formulation and/or in the range of 0.5 to 12% dry weight of a formulation. The preferred UV-absorbents of the embodiments correspond to those comprised of one or more of the glycosylated-acylated-2-phenyl-3′,4′-dihydroxy-chromenylium Meⁿ⁺ group. Although the functionality of the embodiments disclosed herein is not to be construed as being limited to any particular theory, certain copigments stabilize the (i) chromenylium core copigment and enhance UV-absorbency. For example, when (i) chromenylium is linked to (ii) glycosyl, (iii) acyl, and (iv) metalⁿ⁺ copigments, UV-absorbency is enhanced while adding to stability. Furthermore, as used herein, “Meⁿ⁺ UV-absorbent” means an organic UV-absorbent molecule comprised of a plurality of four or more copigments, including (i) a chromenylium-copigment, (ii) one or more glycosyl-copigment, (iii) one or more acyl-copigment, and (iv) one or more Meⁿ⁺ copigment, where n in n+ represents an integer from 2-4, each of which is further defined below.

As used herein, the (i) “chromenylium-copigment” refers to selections from one of the following: 2-(4-hydroxy-3,5-dimethoxyphenyl)chromenylium-3,5,7-triol; 2-(4-hydroxyphenyl)chromenylium-3,5,7-triol; 2-(4-hydroxy-3-methoxyphenyl)chromenylium-3,5,7-triol; and the preferred 2-(3,4-dihydroxyphenyl)chromenylium-3,5,7-triol; 2-(3,4-dihydroxy-5-methoxyphenyl)chromenylium-3,5,7-triol; and 2-(3,4,5-trihydroxyphenyl)-chromenylium-3,5,7-triol. The native (i) chromenylium-copigment is inherently unstable, however each (ii), (iii), and (iv) copigment contributes to the improvement of its stability.

As used herein, the (ii) glycosyl-copigment includes “glycosyl”, “glycosyl_{g}”, “glycoside” and “glycoside_{g}” which refer to the monoglycosyl, oligoglycosyl, and/or polyglycosyl copigments, where g represents an integer from 1-8. For example, of the chromenylium(acylglycosideg) copigments, a monoglycoside, wherein g=1, exhibits up to 10% higher UV absorbance than its corresponding chromenylium copigment. Glycosyl-copigments refer to one or more copigments comprising, for example, the following: hexoses, such as glucose, rhamnose, and galactose; pentoses, such as, xylose and arabinose; oligoglycosyl derivatives, such as rutinose, sophorose, and samubiose; polyglycosyl derivatives; and the like.

As used herein, the (iii) acyl-copigment includes “acyl” or “acylₐ”, which refer to one or more monoacyl, oligoacyl, or PolyAcyl copigments, where a represents an integer from 1-8, preferably from 1-5, and most preferably from 2-5. For example, of the chromenylium(acylₐglycoside) copigments, a monoacyl, wherein a=1, exhibits up to 10% higher UV absorbance than its corresponding chromenylium-glycoside; and a triacyl, wherein a=3, renders up to 50% higher UV absorbance. Acyl-copigments include for example, the following: aliphatic acids, such as acetic, malic, malonic, oxalic, tartaric, and succinic acids; and aromatic acids, generally hydroxycinnamoyl groups bound to one or more glycosides, such as caffeic (Caf), coumaric (Cou), ferulic (Fer), hydroxybenzoic, and sinapic (Sin) acids. The preferred acylglycosyl-copigments are selected from combinations of the aforementioned aliphatic and/or aromatic acid acyl groups; selected from acyl-linked glycoside, biose, triose, tetraose and pentose polymers. In contrast, low potency UV-absorbents, include for example, 2-(4-hydroxyphenyl)chromenylium-3,5,7-triol 3-glucosyl(feruloyl(s)glucoside)-5-(malonylglucoside)s; 2-(4-hydroxyphenyl)chromenylium-3,5,7-triol 3-(maloylglucoside)s; 2-(4-hydroxy-3-methoxyphenyl)chromenylium-3,5,7-triol 3-(maloylglucoside)s; 2-(4-hydroxy-3-methoxyphenyl)chromenylium-3,5,7-triol 3-(succinylglucoside)s; 2-(4-hydroxyphenyl)chromenylium-3,5,7-triol 3-glucosyl(Fer-glucoside)s; 2-(4-hydroxy-3-methoxyphenyl)chromenylium-3,5,7-triol 3-xylosyl(Sin-glucosyl)galactosides, 2-(4-hydroxy-3-methoxyphenyl)chromenylium-3,5,7-triol 3-(Caf-sophoroside)-5-glucosides, 2-(4-hydroxy-3,5-dimethoxyphenyl)chromenylium-3,5,7-triol 3-(p-Cou-rutinoside)-5-glucoside, 2-(4-hydroxyphenyl)chromenylium-3,5,7-triol 3-(Cou-rutinoside)-5-glucosides, 2-(4-hydroxy-3-methoxyphenyl)chromenylium-3,5,7-triol 3-xylosyl(Fer-glucosyl)galactosides, 2-(4-hydroxy-3-methoxyphenyl)chromenylium-3,5,7-triol 3-(p-Cou-rutinoside)-5-glucoside, 2-(4-hydroxy-phenyl)chromenylium-3,5,7-triol 3-(Cou-sophoroside)-5-(malonylglucoside)s, and 2-(4-hydroxy-phenyl)chromenylium-3,5,7-triol 3-xylosyl(Fer-glucosyl)galactosides, ternatins; and the like.

High potency UV-absorbents include, for example, the preferred selections of one or more of 2-(3,4-dihydroxyphenyl)chromenylium-3,5,7-triol 3-(Fer-sophoroside)-5-glucoside; 2-(3,4-dihydroxyphenyl)chromenylium-3,5,7-triol 3-(p-Cou)diglucoside-5-glucoside, 2-(3,4-dihydroxyphenyl)chromenylium-3,5,7-triol 3-(Sin)diglucoside-5-glucoside, 2-(3,4-dihydroxy-5-methoxyphenyl)chromenylium-3,5,7-triol 3-(p-Cou-rutinoside)-5-glucoside, 2-phenylchromenylium-3-xylosyl(Cou(s)glucosyl)galactosides, 2-phenylchromenylium-3-xylosyl(Fer(s)glucosyl)galactosides, and 2-phenylchromenylium-3-glucosyl(Fer(s)glucoside)s; 2-phenylchromenylium-3-xylosyl(Sin-glucosyl)galactosides; 2-(3,4-dihydroxyphenyl)chromenylium-3,5,7-triol 3-(malonylglucoside)s; 2-(3,4-dihydroxyphenyl)chromenylium-3,5,7-triol 3-(succinylglucoside)s; malonylshisonin; petanins; alatanins A, B, C, D, E, F, G. As used herein, highly preferred UV-absorbents include selections from the group of 2-(3,4-dihydroxyphenyl)chromenylium-3,5,7-triol 3-xylosyl(acylglucosyl)galactosides selected from one or more of 2-(3,4-dihydroxyphenyl)chromenylium-3,5,7-triol 3-xylosyl(Cou-glucosyl)galactosides; 2-(3,4-dihydroxyphenyl)chromenylium-3,5,7-triol 3-xylosyl(Sin-glucosyl)galactosides; and 2-(3,4-dihydroxyphenyl)chromenylium-3,5,7-triol 3-xylosyl(Fer-glucosyl)galactosides. Exemplary polyacylglycoside forms of UV-absorbent are selected from one or more of 2-(3,4-dihydroxyphenyl)chromenylium-3,5,7-triol 3-(dimalonylglucoside)s, 2-(3,4-dihydroxyphenyl)chromenylium-3,5,7-triol 3-(maloylsuccinylglucoside)s, 2-(3,4-dihydroxyphenyl)chromenylium-3,5,7-triol 3-(Cou-sophoroside)-5-(malonylglucoside)s, 2-(3,4-dihydroxyphenyl)chromenylium-3,5,7-triol 3-(Fer-sophorosyl)-5-(malonylglucosides), 2-(3,4-dihydroxyphenyl)chromenylium-3,5,7-triol 3-(Sin-Cou)sophoroside-5-(malonylglucoside)s, 2-(3,4-dihydroxyphenyl)chromenylium-3,5,7-triol 3-(Sin-Fer)sophoroside-5-(malonylglucoside)s, 2-(3,4-dihydroxyphenyl)chromenylium-3,5,7-triol 3-(Fer-glucosyl)-6-(glucosyl-Fer)(glucosyl-Cou)glucoside)-5-6-(malonylglucoside); and the like. UV-absorbents may comprise aromatic polyacyl copigments, including those selected from the potent p-hydroxybenzoic, Caf, Cou, Fer, and Sin, such as for example 2-(3,4-dihydroxyphenyl)chromenylium-3,5,7-triol 3-(6″,6″′-diCaf-sophoroside)-5-glucosides; 2-(3,4-dihydroxyphenyl)chromenylium-3,5,7-triol 3-(Caf-p-hydroxybenzoylsophoroside)-5-glucosides; and 2-(3,4-dihydroxyphenyl)chromenylium-3,5,7-triol 3-(6″-Caf-6″′-Fer-sophoroside)-5-glucosides.

As used herein, (iv) Meⁿ⁺ copigment includes “metalⁿ⁺” and “Meⁿ⁺”, which refer to the presence of one or more metals or metalloids in the group of Al(III), B(III), Cd(IV), Cr(II)(III), Cu (II)(III), Ga(III), Ge(IV), Fe(II)(III), Mg(II), Mn(II), Mo(II), Ni(II), V(IV), W(II)(III), and Zn(II), that may form a coordination complex with a suitable UV-absorbent; and preferably where n in n+ represents an integer from 2-4. These copigments are known in the field to bind the 3′,4′-O-dihydroxyl group in the B ring of a flavylium skeleton to form Meⁿ⁺ complexes that shift the equilibrium of the coordinated structures by as much as 20-50 nm in the UV spectrum while further stabilizing them. This shift is further heightened by the previously noted aromatic polyacyl copigments; and particularly as linked to one or more glycosyl copigments, preferably diglycosides, such as sophorosides. As used herein, the Meⁿ⁺ copigment can coordinate only with specific UV-absorbents comprising 3′,4′-O-dihydroxyl groups, such as the preferred 2-(3,4-dihydroxyphenyl)chromenylium-3,5,7-triol, 2-(3,4,5-trihydroxyphenyl)chromenylium-3,5,7-triol, and 2-(3,4-dihydroxy-5-methoxyphenyl)chromenylium-3,5,7-triol, where n in n+ represents an integer from 2-4. In foods, some Meⁿ⁺, such as Al, Cd, Ga, Cr, Ni, and W, are unsuitable; and notably, Cu is omitted in certain embodiments because it tends to oxidize preferred UV-absorbents. Although quadrivalent and trivalent forms may be utilized, divalent Meⁿ⁺ complexes are preferred because, in the case of animal and human administration, the highly preferred copigments are selected from the group of Fe(II) and Zn(II). As used herein, the range of a Meⁿ⁺ copigment is 1 ppb to 70% weight of a dry powder content. In an orally administered liquid formulation, the concentration of Fe in a liquid formulation may be in a suitable range of 1-100 ppb, well below the secondary drinking water standard of 0.3 mg per liter. Preferred salts may be selected from one or more of Meⁿ⁺ ascorbates, Meⁿ⁺ citrates, Meⁿ⁺ malates, where n in n+ represents an integer from 2-4. Selections from FeSO₄·7H₂O, ferrous ascorbate, and ferrous fumarate are preferred sources of Fe. Zinc ascorbates and Zn citrates are preferred sources of Zn in human mineral supplements, particularly for those seeking UV protection; and furthermore, stabilizers may be selected from the group of one or more Meⁿ⁺ organic acid salt, where n in n+ represents an integer from 2-4, such as for example, salts of acetates, ascorbates, citrates, fumarates, gluconates, glutamates, glycinates, lactates, and malates; amines, for example, amino acids, peptides, proteins; and the like. For example, the options for additions of suitable food grade salts include 1 mcg-50 mg ferrous ascorbate, ferrous fumarate, ferrous gluconate, ferrous lactate, ferrous sulfate hydrates, ferrous ammonium sulfate; 1-100 mcg manganese ascorbate, manganese citrate, manganese gluconate, manganese lactate, manganese sulfate; 1-50 mg zinc acetate, 0.01-50 mg zinc ascorbate, zinc bisglycinate, zinc chloride, zinc citrate, zinc gluconate, zinc lactate, zinc phosphates, zinc sulfate. For an orally administered dry tablet or encapsulated mineral supplement formulation, the concentrations of Fe and Zn may be in a suitable range of 0.001-50 mg/dose. The recommended daily iron dosage varies according to gender, e.g., a 10 to 40 mg iron dose for women would be far too high for men whose suitable iron dose may be in the range of 0-50 mcg per dose. For example, in a general dose suitable for both men and women, 10-100 mcg ferrous fumarate was compounded with 500-2500 mg UV-absorbents and was further stabilized by compounding with 10-1000 mg ascorbic acid. The preferred orally administered therapeutic dose of Meⁿ⁺ UV-absorbent mineral supplements is in the range of 0.5 to 12 grams per day, and further, refrigerated storage of Meⁿ⁺ UV-absorbent mineral supplements in the dark is recommended, where n in n+ represents an integer from 2-4.

In certain embodiments, the preferred Meⁿ⁺ copigment is Fe because, after the postprandial uptake of Meⁿ⁺ UV-absorbents, free iron in the blood plasma of animals will form Fe-coordination complexes in such internal environments at physiological pH in the range of 5 to 8, preferably at about pH 7, where n in n+ represents an integer from 2-4. Several Meⁿ⁺ copigments are inherently present in animal cells, blood, plasma, tissues, and in the contents of food, for example Meⁿ⁺, where n in n+ represents an integer from 2-4, may be present in suitable amounts in plants, as well as in tap, well, and spring waters. However, to achieve efficacy with the greatest measure of consistency, exercising the option of formulating with one or more Meⁿ⁺ copigment, where n in n+ represents an integer from 2-4, is preferred.

As used herein, “stabilizer” refers to food grade acids that are requisite to the maintenance of stability of UV-absorbents in animals. The stabilizer may comprise inorganic acids and/or organic acids, however, the preferred stabilizer is comprised of one or more weak organic acid components because, at the same time, one or more organic acid may be selected for its powerful antioxidant properties, as well as for flavor and nutrition. The most highly preferred food grade organic acids are selected from one or more of ascorbic, citric, and malic acids because they serve multiple functions of stabilizer, buffer, and antioxidant. Examples of suitable stabilizers include food grade organic acids selected from one or more of acetates, ascorbates, benzoates, citrates, fumarates, gluconates, glutarates, glycinates, lactates, malates, tartarates, sorbates, succinates, propionates; and food grade inorganic acids such as carbonates and phosphoric acids; and like stabilizers that maintain the efficacy of UV-absorbents by buffering in the acidic range of pH 1-4.5, preferably in the range of pH 2-4.2, most preferably 2-4.1. In certain embodiments the stabilizer is present in the range of 0.1-30% and preferably in the range of 0.1-25% w/w of the total formula dry weight. In liquid solutions, the range of concentrations is diluted, for example, in an aqueous carbonated formula, a beverage may contain phosphoric acid in the range of 25-85 mg/100 ml, preferably in the range of 50-70 mg/100 ml; and carbonic acid in the range of 0.1-1 gm/100 ml, preferably in the range of 0.2 to 0.5 gm/100 ml, in an open container. For example, citric acid may be utilized in the range of 0.1-25%, preferably in the range of 1-20%; and ascorbic acid may be utilized at a lower range of 0.1-5% of the total weight of the formulation. UV-absorbents may be stabilized by some food borne components, and thus the source of the stabilizer may be a fruit juice containing citric acid and/or malic acid; or, for example, 0.5% to 5% acetic acid, such as in the form of vinegars in pickle juices. Thus, one or more of acetic, ascorbic, benzoic, citric, fumaric, gluconic, lactic, malic, sorbic, and propionic acids; mineral salts thereof, and other food grade organic acids maintain the stability of UV-absorbents. The most highly preferred ascorbic acid, Vitamin C, has the added benefit for the reduction of the appearance of photowrinkles, and the preferred Meⁿ⁺ form is a mineral ascorbate, where n in n+ represents an integer from 2-4, such as Mn ascorbate that is beneficial to joints and less irritating to the gastrointestinal tract than the free acid.

As used herein, “CyaCFS” refers to a blend of each of the Meⁿ⁺ UV-absorbents selected from the group of 2-(3,4-dihydroxyphenyl)chromenylium-3,5,7-triol 3-xylosyl(Caf-glucosyl)galactosides; 2-(3,4-dihydroxyphenyl)chromenylium-3,5,7-triol 3-xylosyl(Cou-glucosyl)galactosides; 2-(3,4-dihydroxyphenyl)chromenylium-3,5,7-triol 3-xylosyl(Fer-glucosyl)galactosides; and 2-(3,4-dihydroxyphenyl)chromenylium-3,5,7-triol 3-xylosyl(Sin-glucosyl)galactosides; “CyaCFS Fe” refers to a CyaCFS iron(II) complex; and “CyaCFS Zn” refers to a CyaCFS zinc(II) complex, where n in n+ represents an integer from 2-4. Furthermore, CyaCFS is one of the most efficacious blends of Meⁿ⁺ UV-absorbents.

As the majority of the UV-absorbents occur naturally in plants, the preferred source is from parts of a plant, including fruit, shoot and root. Indeed, for economic feasibility, the source of the UV-absorbent is preferably selected from a superfood crop, such as black currant, purple carrot, red cabbage, purple sweet potato, blueberry, and the like. The UV-absorbents may be isolated by extraction from homogenates of the botanicals in acidified alcohol or acidified aqueous solutions, followed by condensation that results in 20-50% concentrates of the UV-absorbents. For example, in certain embodiments one volume of frozen carrots may be homogenized in an equal volume of ethanol:citric acid (85:15) solution at 40-50° C.; solids may be removed by filtration; alcohol may be removed under vacuum evapo-distillation at 40-50° C.; and yield a product comprised of about 20-50% UV-absorbents with a stabilizer, in this instance, citric acid. The process is undertaken in the dark and the concentrated UV-absorbent is stored at in the dark and preferably below 15° C. Thus, UV-absorbents from plants may further comprise selections of suitable UV-absorbents such as for example alatanins, chrysanthemins, myrtillins, nasunins, ternatins, rutinosides, antirrhinins, tulipanins, quercetin-rutinosides, salvianins, shisonins, kaempferol-malonyl-oligomers, myricetin-malonyl-oligomers, quercetin-malonyl-oligomers; and in the presence of suitable Meⁿ⁺ copigments. The preferred Meⁿ⁺ UV-absorbents are extracts from plant cultivars of the embodiments disclosed herein; specifically those such as CyaCFS are the major Meⁿ⁺ UV-absorbents of the numerous purple to black carrot hybrids, particularly those of D. carota sativus var. atrorubens. Also known as purple-skin hybrids, suitable CyaCFS natural sources include, for example Cosmic Purple, Purple Dragon, Purple Haze, Purple Elite, and Purple Elixir. The highly preferred black carrot sources of Meⁿ⁺ UV-absorbents are pigmented to the core of the carrot root, such as for hybrids including Purple 68, INTA43, Antonina, Black Nebula, Purple Flakke, Deep Purple, and Purple Sun. Concentrates of Meⁿ⁺ UV-absorbents were selected from commercial sources based on the qualities, quantities, and costs of copigments. For example, Exberry@ products as used in formulations of the embodiments disclosed herein include Burgundy GNT 181531; Red GNT 153330; Bordeaux GNT 181524; and Purple Plum GNT 18240041. In exemplary cases of economic feasibility of less than US $1 per gram of Meⁿ⁺ UV-absorbents, Exberry® food grade products as used herein, were comprised of blends of botanical extracts selected from the group of carrot, blueberry, and blackcurrant, typically comprising Meⁿ⁺ UV-absorbents in the range of 20-50% dry weight contents, where n in n+ represents an integer from 2-4.

As used herein, a suitable range of dosages for a 30-100 Kg person of one or more Meⁿ⁺ UV-absorbents in a prophylactic formulation for the systemic protection against damaging effects of UV radiation to the skin of an animal is 0.5 to 12 gm per day, where n in n+ represents an integer from 2-4. The dose for animals other than human may be determined by fractions or multiples of 0.01-0.03 gm/Kg/day, based on the average weight of the animal to be treated. Formulations preferably are stored in the dark and furthermore are kept preferably in containers impervious to light.

As used herein, “solar radiation” means broad spectrum sunlight and includes visible light as well as infrared and ultraviolet (UV) radiation. As used herein, “UV” radiation (100-400 nm) includes UVC, in the wavelength range of 100-280 nm; UVA, in the wavelength range of 315-400 nm; and UVB, in the wavelength range of 280-315 nm, high energy radiation that is particularly deleterious to the skin. While UVA is involved in the generation of peroxynitrates (ONOO⁻) and superoxide anions (O₂*⁻), UVB has long been known in the field for high energy photocarcinogenicity.

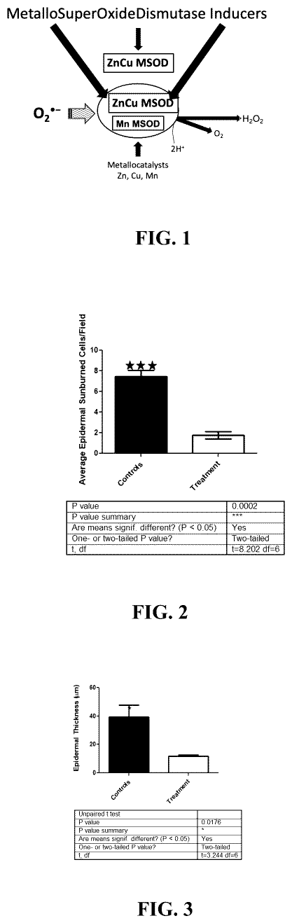

As used herein, “CPD” refers to cyclobutane pyrimidine dimers and “8-oxo-dG” refers to 8-oxo-2′-deoxyguanosine, well-known in the field as DNA damage and oxidation products that arise from exposure to photocarcinogenic UVB. Thus, when an animal is exposed to UVB, it may be absorbed by DNA, causing the formation of CPD+ and 8-oxo-dG+, foreboding indicators of genetic mutations in cancer. It is for this reason that UVB is the particular photocarcinogen of concern in embodiments disclosed herein. Therefore, as used herein, “photocarcinogen” includes UV radiation that, upon exposure, induces genetic mutations in cancer cells of animals, including humans. Furthermore, when a treated animal is exposed to UV radiation, DNA damage is averted by absorbance of UV radiation by the formulations of the embodiments disclosed herein. As used herein, “MSOD” refers to one or more of the metalloenzymes, metallosuperoxidedismutases. Without being limited to any particular theory of functionality, it is believed that the instant formulations are effective in their ability to mitigate injuries from photocarcinogens by absorbing UV, on the one hand, and stimulating metallosuperoxidedismutases to scavenge oxygen radicals such as ONOO⁻ and O₂*⁻, on the other hand. This is accomplished in a manner that previously had not been applied, particularly for the mitigation or prevention of the effects of exposure to photocarcinogens, that cause injury manifested by, for example, epidermal sagging, cell death, precancerous lesions, and/or cancerous DNA mutations. Indeed, the formulations naturally absorb UV rays while internally stimulating the production of agents that may protect against genetic damage. It is believed that one of the mechanisms of action on exposure to a photocarcinogen is to systemically reduce damage to DNA and modulate generation of deleterious free radicals. Embodiments thus provide dual-acting complimentary ingredients to provide protection from UV damage by absorbing the radiation before it reaches the DNA and powerful antioxidant activity. As illustrated in FIG. 1, the mode of action of MSOD is to dismutase the O₂*⁻ and, MSOD are known in the art as the first line of defense against negatively charged free radicals formed via electron donation to oxygen. FIG. 1 shows the reactions and transformations of the superoxide anion, O₂*⁻. The dismutation of O₂*⁻ by MSOD generates oxygen (O₂) and hydrogen peroxide (H₂O₂). Thus, administration of the instant formulations comprising one or more MSOD-inducer promotes the dismutation of O₂*⁻, that further protects against formation of peroxynitrates (ONOO⁻). The MSODs in mammals, Mn MSOD and ZnCu MSOD, may be activated in vivo by manganese, zinc, and copper. In mammals, such as in humans, the MSOD are, for example, “ZnCu MSOD” is catalyzed by Zn and Cu ions; and “Mn MSOD” is catalyzed by Mn ions. The MSOD is catalyzed by the group of Meⁿ⁺, Mn, Zn, and Cu, and these ions may be supplied from outside of the body and, when made available according to embodiments disclosed herein, contribute to the structures and functions of MSOD. As used herein, “ONOO” refers to peroxynitrates and “O₂*⁻” refers to superoxide anions. The metallocatalytic dismutation reaction is extremely efficient, but in the absence of Meⁿ⁺, O₂*⁻ and ONOO* reactive species can generate even more radicals in abundance, where n in n+ represents an integer from 2-4. As used herein, “antioxidant” refers to the ability of a chemical to act as an electron donor and, thereby, neutralize free radicals. UV-absorbents of the formula are in fact powerful antioxidants. Indeed, by controlling O₂*⁻ via dismutase activity, the rate of antioxidant activity by UV-absorbents of the embodiments is the key to mediation of the concentration of radicals in tissues, cells, and organelles, especially through the endoplasmic reticulum and mitochondria inside of human cells.

As used herein, “inducer” refers to a “MSOD-inducer” meaning one or more bioactive substances in a formulation of embodiments disclosed herein that decreases reactive species that would otherwise cause inflammation and oxidative stress. Inducers are optional components of the formulations of the embodiments disclosed herein. One skilled in the art may formulate the instant formulation with an inducer, preferably with one or more selection from the group of flavins. As used herein, “flavin” may be an inducer selected, for example, from flavin mononucleotides, flavin adenine dinucleotides, lumiflavin, niacins, niacinamides, nicotinamide adenine dinucleotides; and preferably from riboflavins selected from one or more of riboflavin, vitamin B₂, and their phosphates including the preferred riboflavin Na phosphate. Optionally, one or more flavins may be formulated in the instant formulation in the range of 1-100 ppm and in the preferred range of 5-20 ppm. For example, riboflavin Na phosphates may be preferably in the range of 5-20 ppm. Inducer options may include one or more ammonium compound, rebaudiosides, sugars, alcohols, flavonoids, flavonols, and proanthocyanidins. Furthermore, proanthocyanidins may be selected from one of more procyanidins, prodelphinidins, propelargonidins, resveratrol, catechins, epicathechins, and epigallocatechins, in the range of 0.5 to 12 grams per dose. In certain embodiments, the inducers are effective in decreasing reactive species that otherwise would cause inflammation and oxidative stress in the animal to whom the formulation is or is to be administered in the range of 1 ppm to 50%, and preferably in the range of 100-6000 ppm, in liquid formulations that are applied 1 to 3 times daily. As used herein, “ammonium compound” refers to optional components of inducers that are food grade compounds selected to further enhance MSOD. Ammonium compounds, for example, may be selected from one or more ammonium salts, such as ammonium sulfate, C₄₂H₆₅NO₁₆ and NH₄HCO₃, administered in the range of 1 to 100 ppm.

As used herein, the effective human dose is in the range of 0.5-12 gm per day of UV-absorbent for the average 30-100 Kg human. To achieve this dosage, it may be administered 1 to 5 times daily. The recommendation for multiple dosage per day is based on uptake of the UV-absorbent to the blood plasma after approximately 6-11 hours following each oral administration. Therefore, in certain embodiments, protection from UV exposure is achieved when there is some overlap of consumption to its peak in the blood plasma of the animal to whom the formulation is administered. Thus, pre-exposure prophylactic daily doses for up to a week prior to exposure allows for accumulation in skin tissues. The general amount of said UV-absorbent is in the range of 0.1%-70% of the formula; the amount of said stabilizer is 0.1-30% of the formula; and wherein the therapeutically effective amount of one or more starter doses is in the range of 3 to 12 grams total UV-absorbent dry weight, as taken 6-24 hours prior to exposure to a photocarcinogen; and wherein said therapeutically effective amounts of one or more maintenance dose is in the range of 0.5 to 3 grams UV-absorbent per day, as taken for 3-7 days or more prior to exposure to a photocarcinogen; and/or on the next day after the starter dose, taking 0.5 to 3 grams UV-absorbent dose per day for the days, weeks, or months of exposure to a photocarcinogen. For convenience, suitable dosage amounts are a starter dose in the range of about 6 to 24 hours before exposure, followed the next day with a maintenance dose, and then taking daily maintenance doses for a week or more to cover outdoors activity in sunlight, or other exposure to UV radiation such as from a tanning bed. The preferred route of administration is oral and this includes drinking by mouth, sublingual absorption, eating a serving, or swallowing a pill. A dose may be taken before, during or after a meal, with preference for pre-prandial oral administration.

The formulations may be contained in a suitable container or vessel, such as in a bottle, can, box, envelope, metallic mylar foil sealable bags, aluminum foil lined stick, and the like, preferably packaged in a container that is impervious to light. Animal hydration vessels, such as bottles, pans, dishes and troughs, may be covered to exclude light with, for example metal lids or aluminum foil wraps, and, thus, prevent premature photodecay of the components. In certain embodiments the formulation may be compounded into prepared foods, such as for example, pastries, such as cakes, confectionary, cookies, crackers, croissants, wafers, cereals, granola, candies, chocolates, bobas, brownies, ice creams, gelatins, parfaits, puddings, yoghurts, energy bars; and further into edible decorations, flavorings, frostings, sauces, soups, and syrups. In certain embodiments the formulation may be compounded as a dry powder (e.g., dry powder electrolyte, hydration, antioxidant, and drink mix), pill, capsule, gelcap, tablet, lozenge, gummy, melt, oral strip, sublingual drop, micelles, and etc.

As used herein, “excipients” means optional additives that include inactive ingredients, such as for example alcohols, artificial colors, artificial flavors, artificial sweeteners, sugars, natural sweeteners, binders, caffeines, carrageenans, chelants, diluents, drying agents, fiber, flavors, gums, juices, MSG, maskers, minerals, mucopolysaccharides, colorants, dyes, flavors, phenolic compounds, pH-components (acids and bases), potassium, phosphates, salts, Na, spices, starch, minerals, vitamins, and the like. In some embodiments, excipients are pharmaceutically acceptable excipients. Excipients in dry formulations may include anticaking and flow agents, such as dipotassium phosphates, celluloses, and silicone dioxides in the range of 1-10% weight of, for example a dry powder formula. Further, excipients may include sweeteners to balance the sour flavor of the aforementioned acid components, suitable sweeteners selected from sugars and high intensity sweeteners that are generally effective when applied in the range of 1 ppm-50%. For example, sweeteners may be selected from sugars, such as allulose, beet sugars, cane sugars, corn sugars, dextrose, fructose, honey, mannose, sucrose, and xylose; sugar alcohols, such as erythritol, xylitol, and mannitol; high intensity sweeteners, such as acesulfame-K, aspartame, neotame, Sucralose®, saccharin; and botanicals, such as Stevia and its components, for example steviosides, steviobiosides, dulcoside A, rubusosides, rebaudiosides, rebaudioside A, rebaudioside B, rebaudioside C, rebaudioside D, rebaudioside F, and rebaudioside M; formulated in an effective range of 1-500 ppm; and in a preferred range of 50-300 ppm. Any of the high intensity sweeteners, such as steviosides or Sucralose® may be applied in combination with other sweeteners, such as fructose, cane sugar, caramels, corn syrups, agave syrups, glucose, honey, maltose, molasses, sophoroses, and/or sucrose in the range of up to 50% of the formula. In another embodiment, alcohols, such as ethanol, may be the preferred carriers that enhance solubility and accelerate uptake of UV-absorbents. Excipients may further include selections of flavor components, such as for example natural and artificial flavors in the range of 0.1%-50%, and in the preferred range of 1%-25%. Excipients may include a carrier, which may be of pharmaceutical grade.

In certain embodiments, one or more or all of the components of the formulation are food grade compounds. In certain embodiments, one or more components of the formulation are pharmaceutical grade compounds. In certain embodiments, components of the formulation are either a food grade or a pharmaceutical grade component.

As used herein, “oral administration” refers to swallowing, imbibing and otherwise drinking an aqueous or dry formulation, and includes sublingual administration, as well as taking an encapsulated or bulk powder by mouth. In certain embodiments the formulation is non-toxic to humans and has a pleasant taste. In certain embodiments, the methods include the oral administration to an animal in need of therapeutically effective amounts of formulations that are effective in absorbing radiation in the UV-spectrum, preferably in the UVA and UVB range. In certain embodiments, the methods include the oral administration to an animal in need of therapeutically effective amounts of Meⁿ⁺ UV-absorbent formulations that also may be effective mineral supplements.

In certain embodiments, the formulation is a potable carbonated beverage. Suitable levels of carbonation range from about 2 to about 5 volumes of dissolved carbon dioxide, preferably about 3.5 to about 5 volumes of dissolved carbon dioxide. Suitable autogenous pressure within the container containing the carbonated beverage may range up to about 100 psi and is typically about 60 psi. Suitable carbonated beverages include soft drinks sparkling waters, colas, sodas, seltzers, hard seltzers, and carbonated fruit juices Carbonation protects against oxidation and beverages containing carbonation are preferably processed in the dark and stored in containers that are impervious to light.

As used herein, “topical” and “topical application” include directly laying on, spraying, spreading or wiping on outer skin or scalp, but excluding eyes, e.g., by use of hands or by use of applicators, such as a wipe, roller, tissue, or spray.

As used herein, “sunblock” refers to formulations that may be in the form of, for example, a gel, cream, lotion, spray, stick, and like topical products that block, absorb, screen, or reflect some UV, offering temporary surface-blocking of exposure to the sun. Furthermore, “sunblock composition” includes formulations that physically block radiation, mainly comprised of zinc or titanium. As opposed to a conventional sunblock that shades patches of skin to which it is applied superficially, embodiments disclosed herein work systemically to protect against and/or mitigate and/or prevent injury from photocarcinogens, and the instant methods and formulations are systemic internal defenders against problems that arise from exposure to radiation, such as from the sun, via oral routes of administration, that have not been achieved previously.

As used herein, “storage” refers to the conditions of the location for keeping the product fresh for the duration that it sits prior to consumption. In certain embodiments, the active formulation is stored and/or transported in a dry dark environment at temperatures in the range of 1-30° C., and preferably in the range of 5-20° C. As used herein, “processing and storage in the dark” refers to undertaking both manufacture and storage in the dark, with allowance for short duration of artificial light exposure of less than 200-250 μmol m⁻²s⁻¹ for no more than a total accumulated exposure time of 1-5 minutes under temperatures in the range of 1-30° C., and preferably in the range of 5-20° C. Dry formulations may be stored in a freezer below 0° C. Processing and storage in the dark is critical to maintaining efficacy of the ingredients. Some components of the formulation are rapidly photooxidized by light, that is, within 5-10 minutes at temperatures above 50° C.; therefore, manufacture and storage in a cool dark environment is essential.

As used herein, “prevention of photowrinkling” refers to benefits to skin health based on reduction of photocarcinogenic skin thickening, skin sagging, cell death, and other damage, and includes partial or complete mitigation or amelioration of the appearance of skin wrinkling, skin creasing, skin thickening, skin sagging and/or fine lines due to external damage such as from UV exposure from, for example, the sun or a tanning bed.

In certain embodiments, the technology of the embodiments disclosed herein is compatible with other medical treatment methods and the formulation further are applicable in conjunction with vaccines therapies, such as for example, as an adjuvant treatment for melanoma risk in combination with, for example, personalized mRNA-4157-P201 cancer vaccines and/or pembrolizumab-type therapies. In this case, the formula may be adjusted to allow for a hypodermic injection.

Embodiments disclosed herein relate to UV-absorbents and other components in formulations that protect animals, including humans, from damage and photocarcinogenesis caused by exposure to the UV spectrum of sunlight. This category of systemic protection from sunburn is different from topical sunblock cosmetics and, therefore, is distinguished by classification as a photocarcinogen mitigator. The methods and formulations of embodiments disclosed herein achieve systemic protection against photocarcinogenesis by reducing injury to the skin and damage to DNA. Furthermore, by formulating antioxidants, the methods and formulations modulate the formation of peroxynitrates (ONOO*) and superoxide anions (O₂*⁻) that would otherwise generate even more deleterious radicals. As used herein, “superfoods” refers to high nutrient density foods, preferably high in antioxidants, considered to be especially beneficial for health and well-being.

In certain embodiments, an aqueous solution formulation has a finished Brix soluble solids content in the preferred range of about 0.05%-70%, and may be safely administered to a host, subject or patient in need thereof daily as an oral formulation, one to three times per day.

In certain embodiments, the formulations comprising Meⁿ⁺ UV-absorbents are processed and stored in the dark, with minimized duration of exposure to light to prevent loss of activity from photooxidation of one or more of the compounds, where n in n+ represents an integer from 2-4. In certain embodiments, a container used to house the formulation is impervious to light.

In certain embodiments, the formulation may be bottled using a hot filling process. For example, the process may comprise: filling containers with the formulation at a fill temperature in the range of about 83°-95° C., maintaining a set headspace of about 1.3 cm and covering with a suitable cap; inverting the container and holding it in the inverted state at a temperature in the range of about 83°-90° C. (or higher) for at least about 6 seconds, preferably for about 60 seconds or longer; and followed by turning the container right-side-up and allowing it to air cool in the dark. Preferably the hold time is a minimum of 6 seconds at 83° C. (or a higher temperature for longer) to ensure safety of the contents.

The following Table 1 lists exemplary UV-absorbent compounds in accordance with certain embodiments.

TABLE 1
Examples of UV-absorbents (Abbreviation)
In the Tables and Examples of the embodiments that follow,
the headings, Cya, Del, Pel, Peo, Pet, Mal, and PolyAcyl,
refer to their corresponding groups of each heading of
exemplary UV-absorbents. UV-absorbents cited in the
exemplary formulations of the embodiments are
selected from one or more of the
compounds listed.

Cya
2-(3,4-dihydroxyphenyl)chromenylium-3,5,7-triol 3-xylosyl(Fer-glucosyl)galactoside (CyaFer)
2-(3,4-dihydroxyphenyl)chromenylium-3,5,7-triol 3-xylosyl(Sin-lucosyl)galactoside (CyaSin)
2-(3,4-dihydroxyphenyl)chromenylium-3,5,7-triol 3-xylosyl(Cou-glucosyl)galactoside (CyaCou)
2-(3,4-dihydroxyphenyl)chromenylium-3,5,7-triol 3-xylosyl(Caf-glucosyl)galactoside (CyaCaf)
2-(3,4-dihydroxyphenyl)chromenylium-3,5,7-triol 3-malonylglucoside (CyaMal)
2-(3,4-dihydroxyphenyl)chromenylium-3,5,7-triol 3-(p-Cou)diglucosyl-5-glucoside
2-(3,4-dihydroxyphenyl)chromenylium-3,5,7-triol 3-(Fer-sophoroside)-5-glucoside
2-(3,4-dihydroxyphenyl)chromenylium-3,5,7-triol 3-(maloylsuccinyl)glucosides
Del
2-(3,4,5-trihydroxyphenyl)chromenylium-3,5,7-triol 3-(p-Cou-rutinoside)-5-glucoside (DelCou)
Pel
2-(4-hydroxyphenyl)chromenylium-3,5,7-triol 3-xylosyl(Fer-glucosyl)galactoside
2-(4-hydroxyphenyl)chromenylium-3,5,7-triol 3-(Cou)rutinoside-5-glucoside
Peo
2-(4-hydroxy-3-methoxyphenyl)chromenylium-3,5,7-triol 3-xylosyl(Fer-glucosyl)galactoside
2-(4-hydroxy-3-methoxyphenyl)chromenylium-3,5,7-triol 3-xylosyl(Sin-glucosyl)galactoside
2-(4-hydroxy-3-methoxyphenyl)chromenylium-3,5,7-triol 3-(Caf-sophorosy1)-5-glucosides
2-(4-hydroxy-3-methoxyphenyl)chromenylium-3,5,7-triol 3-(p-Cou-rutinoside)-5-glucoside
Pet
2-(3,4-dihydroxy-5-methoxyphenyl)chromenylium-3,5,7-triol 3-(p-Cou-rutinoside)-
5-glucoside (PetCou)
Mal
2-(4-hydroxy-3,5-dimethoxyphenyl)chromenylium-3,5,7-triol 3-(p-Cou-rutinoside)-5-glucoside
PolyAcyl
2-(3,4-dihydroxyphenyl)chromenylium-3,5,7-triol 3-(dimalonylglycosyl)glucosides
2-(3,4-dihydroxyphenyl)chromenylium-3,5,7-triol 3-(Cou-sophorosy1)-5-(malonylglucoside)s
2-(3,4-dihydroxyphenyl)chromenylium-3,5,7-triol 3-(Fer-sophorosy1)-5-(malonylglucoside)s
2-(3,4-dihydroxyphenyl)chromenylium-3,5,7-triol 3-(Sin-Cou)sophorosyl-5-malonylglucosides
2-(3,4-dihydroxyphenyl)chromenylium-3,5,7-triol 3-(Sin-Fer)sophorosyl-5-malonylglucosides
2-(3,4-dihydroxyphenyl)chromenylium-3,5,7-triol 3-(Fer-glucosyl)-6-(glucosyl-Fer)(glucosyl-
Cou)glucoside)-5-6-(malonylglucoside)
2-(4-hydroxyphenyl)chromenylium-3,5,7-triol 3-(Cou-sophorosyl)-5-malonylglucosides

The following Table 2 lists suitable approximate ratios of components of the formulation. Suitable stabilizers may be selected from one or more of acetic, ascorbic, citric, succinic, gluconic, glutaric, fumaric, lactic, malic, tartaric, sorbic, benzoic, propionic, carbonic, and phosphoric acids; and like stabilizers that maintain the integrity of UV-absorbents and inducers.

TABLE 2
Exemplary ratios of one or more of the
major components for oral formulations

| | Suitable Range | Preferred Range |
|---|---|---|
| Composition A | | |
| UV-absorbent | 0.1-70% | 1-12% |

Suitable ratios of one or more of the following components

| | | |
|---|---|---|
| Cya | 10-90% | 50-90% |
| Del | 0.1-20% | 0.1-15% |
| Pet | 0.1-20% | 0.1-15% |
| PolyAcyl | 0.1-20% | 0.1-15% |
| Composition B | | |
| UV-absorbent | 0.1-70% | 1-12 |
| Stabilizer | 0.1-30% | 10-25% |
| Composition C | | |
| UV-absorbent | 0.1-70% | 1-12% |
| Stabilizer | 0.1-30% | pH 2-4.1 |
| Excipients, Inducers, Meⁿ⁺ | (Optional) | 0.1-90% |

The following Table 3 provides exemplary components of oral formulations.

TABLE 3
General Aqueous Oral Formula

| Composition | Suitable Range | Preferred Range |
|---|---|---|
| UV-absorbent | 0.1-70% | 1-12% |

Suitable ratios of one or more of the following components

| | | |
|---|---|---|
| CyaFer Meⁿ⁺ | 10-55% | 20-50% |
| CyaSin Meⁿ⁺ | 10-55% | 20-50% |
| CyaCou Meⁿ⁺ | 1-10% | 1-5% |
| CyaMal Meⁿ⁺ | 1-10% | 1-5% |
| PetCouMeⁿ⁺ | 1-10% | 1-5% |
| DelCou Meⁿ⁺ | 1-10% | 1-5% |
| Stabilizer | 0.1-30% | pH 2-4.2 |
| Organic acids | | |
| Meⁿ⁺ organic acid salts (Optional) | | |

While the embodiments described include a limited number of embodiments, these specific embodiments are not intended to limit the scope as otherwise described and claimed herein. Modification and variations from the described embodiments exist. The following examples are given as a specific illustration of embodiments disclosed, and it should be understood that the embodiments disclosed are not limited to the specific details set forth in the examples.

### Example 1

Formulations for oral administration were evaluated through multiple pre-clinical models of human diseases based on methods (Carpenter et al., 2022) modified by A. Indra and G. Ganguli-Indra (see full citations below) to develop the treatment protocols for the evaluation of HB7 (defined below); and who thereby established a novel role for an orally administered formulation in the prevention of solar UV-induced aging and skin cancer. The following studies were aimed at formulations that absorb UV and boost the most powerful reactive oxygen and reactive nitrogen defense mechanisms in the human body. Formulation HB7 (set forth below) comprises a blend of UV-absorbents comprising Exberry® Bordeaux and Exberry® Red, that included CyaCFS; stabilizers comprising citric acid, Na citrate, Mn citrate, and Zn citrate in the range of pH 1-4.5; and optional inducers comprising riboflavin, riboflavin Na phosphate, NH₄HCO₃, and rebaudiosides; Brix, in the range of 0.2-0.4; stored in the temperature range of 10-20° C., in a dark room. The methods and formulations of the components in the orally administered liquid HB7 formulation are given below.

Characterization of the in vivo role of orally administered solutions was undertaken to show the in vivo effects of HB7 on UV-induced degradation of the extracellular matrix, as follow: I. Determine in vivo effects of a systematic orally administered HB7 test solution on UV-induced sunburn and suntan.

Protocol: Cohorts of adult mice (B/6 or SV/129) were fed with up to approximately 10 ml/day of either the HB7 liquid formulation or regular water (n=4/group) for 5 weeks prior to UVB radiation. Post-feeding, all mice were exposed to a single acute dose of UVB 160 mJ/cm² that was sufficient to induce sunburn and DNA damage on skin. Tissue samples of mouse skin were harvested at 24 hours post-UVB radiation. Histological analyses via post-hematoxylene and eosin (H&E) staining were performed on formalin-fixed paraffin-embedded skin sections from mice treated with or without HB7 to determine the % SBC with the characteristic morphology consisting of a pyknotic nucleus and eosinophilic cytoplasm based on methods (Carpenter et al., 2018) that were modified by A. Indra and G. Ganguli-Indra to develop the treatment protocols for the evaluation of HB7. In addition, Fontana Mason staining for determining pigmented melanocytes and corresponding melanin distribution was performed. Paraffin skin 5 μm sections were stained with Orcein-Goldner's trichrome stain to highlight collagen fibers, elastic fibers, and blood vessels. Histological examinations of tissue sections were performed using a Leica DMR microscope.

Results: As compared to water controls, the results indicated the following: In FIG. 2, a statistically significant reduction of the formation of sunburn cells in murine skin following treatment with HB7 (significantly reduced formation of sunburn cells in murine skin following treatment with HB7 (Treatment, right) as compared to Controls (left); and in FIG. 3, which shows significantly reduced epidermal thickness in murine skin following treatment with HB7 (Treatment, right) as compared to Controls (left).

The results showed clear evidence of absorbance of UV radiation by HB7 that resulted in significantly reduced UV-degradation of the extracellular matrix in vivo. The effects of sunburn were significantly reduced 24 hours post-UVB radiation.

II. The effects of HB7 oral solutions on UV radiation induced DNA damage were conducted in vivo to determine if the ingredients were effective UVB-absorbents and mitigated UVB-induced (1) formation of cyclobutane pyrimidine dimers (CPD+) and (2) 8-oxo-2′-deoxyguanosine (8-oxo-dG) oxidative stress.

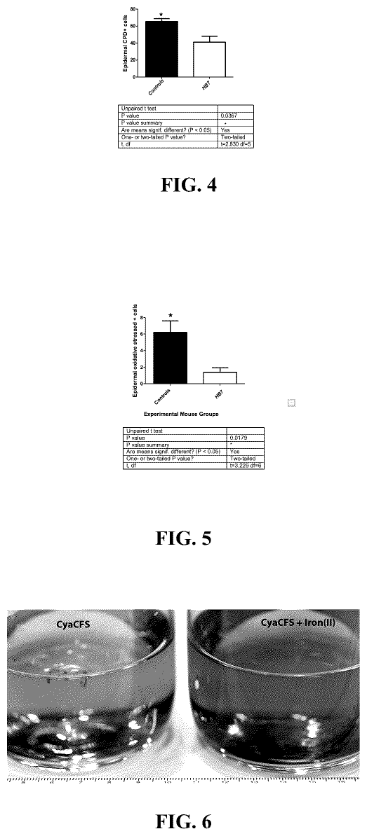

Protocol: The effects of oral administration of HB7 on UVB-induced DNA damage were analyzed on skin biopsies from either test HB7 or regular water fed mice by using the following endpoints: Induction of CPD+ and induction of 8-oxo-2′-deoxyguanosine (8-oxo-dG)- the major form of oxidative DNA damage. CPDs were detected by immunostaining with a CPD-specific antibody (Cosmo Bio USA, 2792 Loker Avenue West, Suite 101, Carlsbad, CA) after UV-exposure. For 8-oxo-dG, water controls or HB7-treated murine skin were immuno-stained with an 8-oxo-dG-specific antibody (Trevigen, Gaithersburg, MD) after UV-exposure. Result: Mice fed with HB7 showed absorbance of UV radiation that resulted in reduced formation of UV-induced DNA damage in vivo including a significant (p=0.0367) reduction of CPD+ and 8-oxo-dG+ in oxidatively stressed epidermal cells in the murine skin, as compared to that found in control mice fed with regular water. A graphic summary of reduced formation of UV-induced CPD+ is presented in FIG. 4, which shows significantly reduced cyclobutane pyrimidine dimers (CPD+) formation and oxidative stress following treatment with HB7 (HB7, right) as compared to Controls (left). UV radiation induces generation of reactive oxygen species that cause damage to DNA, RNA, proteins and lipids. On irradiation of cellular DNA, photo-oxidation reactions with DNA caused the formation 8-oxo-dG and other species in controls. As a result of UV radiation post-feeding treatment with HB7 as compared to water-fed controls, highly significant (p=0.0179) reductions of 8-oxo-dG+cells were observed, as graphically summarized in FIG. 5, which depicts a statistical summary showing highly significant reductions of 8-oxo-dG+cells, indicative of prevention of oxidative stress damage to DNA, RNA, protein and lipids, as a result of UV radiation post feeding treatment with HB7 (HB7, right) as compared to Controls (left). Statistical Analyses: Images and data were analyzed and quantified using ImageJ software (NIH). Multiple IHC fields on each slide from all groups were randomly selected and 20 fields per group were counted. Populations were based on water-fed control mice, n=4; and HB7-fed mice, n=4. Slides were analyzed independently in a double-blinded manner by investigators, while means and statistical significance (p) were calculated using GraphPad Prism software at p<0.05, as determined by Student's 2-tailed T-test.

Overall Results: The orally administered solutions in accordance with embodiments disclosed herein prevented solar UV-induced photowrinkling and skin cancer, as follows: The cellular mode of action as compared to control skin homeostasis showed that oral administration of HB7 significantly modulated proliferation and cell survival. Absorbance of UV in the skin of treated mice prevented UVB-induced DNA damage and sagging attributable to degradation of the extracellular matrix.

Conclusions: Pre-clinical in vivo studies indicated that feeding mice with HB7 rapidly protects against the deleterious effects of solar radiation including the prevention of damage to DNA and formation of sagging, photowrinkled skin. Oral administration of HB7 significantly reduced acute solar UV-induced formation of sunburn cells in murine skin and HB7 significantly reduced acute solar UV-induced skin thickening in murine skin within 24 hours after exposure to UV-radiation. Moreover, HB7 significantly reduced acute solar UV-induced damage to DNA based on the observed reduced formation of CPD and 8-oxo-dG in the murine skin, and oral administration of HB7 provided protection from the photocarcinogen. The stability of the active ingredients as they coursed through the digestive tract, into the circulatory system, and to the targeted tissues, is indicated by the efficacy data.

Components of the Orally Administered Liquid HB7 Formulation

The novel formulations of embodiments disclosed herein have the following integral components for the treatment of a mouse: Meⁿ⁺ UV-absorbent and stabilizer; and further included optional components such as excipients and inducers. The HB7 formulation comprising the components was dissolved in 10-500 ml water for oral administration, as follow:

| HB7 Composition | Sample |
|---|---|
| UV-absorbent | 500 ppm |
| Stabilizer | 2000 ppm |
| Citric Acid | |
| Mn citrate | |
| Na citrate | |
| Zn citrate | |
| pH | 2.7 |

Excipients and Inducers (Optional)

| | |
|---|---|
| Cherry flavor | 500 ppm |
| Lime flavor | 500 ppm |
| Water | |
| Flavins | 3 ppm |
| Ammonium compound | 5 ppm |
| Stevia | 250 ppm |

### REFERENCES

Carpenter E L, Wyant M B, Indra A, Ito S, Wakamatsu K, Merrill G F, Moos P J, Cassidy P B, Leachman S A, Ganguli-Indra G, Indra A K. (2021) Thioredoxin Reductase 1 Modulates Pigmentation and Photobiology of Murine Melanocytes in vivo. J Invest Dermatol. S0022-202X (21)02605-1.
Carpenter E L, Le M N, Miranda C L, Reed R L, Stevens J F, Indra A K, and Ganguli-Indra G. (2018) Photoprotective Properties of Isothiocyanate and Nitrile Glucosinolate Derivatives from Meadowfoam (Limnanthes alba) against UVB Irradiation in Human Skin Equivalent. Front Pharmacol. 9:477.

### Example 2

Exemplary compositions were formulated for oral administration. The recommended daily dose is 0.1-2.5 L, 1 to 3 times per day for a week or more, and preferably 2 to 3 daily applications for 1-7 days prior to and during exposure to sunlight.

TABLE 4
Exemplary Oral Formulations
Efficacious Dry Powder Formulation for Single-Serve Packets

| | |
|---|---|
| Suitable Range | Preferred Range |

| Meⁿ⁺ UV-absorbent | | | | |
|---|---|---|---|---|
| Exberry ® Purple Plum | 1.5-36 | gm | 3-18 | gm |
| Stabilizer | | | | |
| Ascorbic acid | 0.01-1 | gm | 0.01-0.1 | gm |
| Citric acid | | | pH 2-4.2 | |
| Meⁿ⁺ (Optional): | | | | |
| Mn | 0-100 | ppb | 5-50 | mcg |
| Zn | 0-5000 | ppb | 1-5 | mcg |
| Fe | 0-100 | ppb | 10-50 | mcg |

Dry powder doses for humans are packaged for individual single servings in light impervious mylar foil packets. Exemplary packets each contain one serving of 3 to 15 grams of dry powder. Stir the powder blend into 6-12 ounces of water. At 1-3 servings per day, after 3-7 days, go out into the field to enjoy the sunlight. For a quick start, 6 to 24 hours prior to UV exposure, take a starter dose of 9-36 gm dry weight content. The starter may be taken all at once, as dissolved in water, juice, tea, or alcohol beverages; and preferably as one serving at night around bedtime. About a third of the dry weight of Exberry® Purple Plum is comprised of Meⁿ⁺ UV-absorbents.

### Example 3

A series of formulations for oral administration were made from dry powder concentrates that, for administration, were dissolved in water for drinking by the user. The powder, including excipients, such as sugar and other flavors, may be packaged for individual applications in the preferred range of 5-30 gm/dose. The dry powder is diluted in 0.1-2.5 L water, juice, tea, or beverage such that the aqueous oral formulations are safely administered daily, one to five times per day, to an animal, particularly to a human. Oral administration may be administered by drinking by mouth, and sublingually, for systemic delivery of ingredients. See, for example, Table 1, for preferred UV-absorbent abbreviations, and Table 5 for exemplary dry powder concentrates.

TABLE 5
Exemplary dry powder concentrates for dilution in water
for oral administration of a single serving.
Exemplary Meⁿ⁺ UV-absorbent Dry Formula

| | Suitable | Preferred |
|---|---|---|
| Composition | Range/Serving | Range/Serving |
| Meⁿ⁺ UV-absorbent | 0.5-12 grams | 1-12 grams |
| Stabilizer | 0.01-1 gram | pH 2-4.2 |

Stabilizers may include organic acids, such as acetates, ascorbates, citrates, gluconates, malates; and/or inorganic acids, such as phosphoric acids and carbonic acids; and Meⁿ⁺ salts, thereof, in the range of 0.01-1 gram per serving. Excipients may include artificial sweeteners, binders, buffers, flow agents, anticaking compounds, thickeners, diluents, dipotassium phosphate, fiber, flavors, gums, inducers, juices, maskers, natural colors, natural flavors, proanthocyanidins, macha, tea, salt, silicone dioxides, Na, sugars, and/or sweeteners.

Daily servings are administered orally 1 to 5-times per day, each 5-30 gm of the formula dissolved in 0.25-2.5 L water for 14 days or more, and for convenience, a starter dose may be administered a day prior to exposure to ONOO⁻ and 02*⁻ generating factors.

Exemplary Dry Blend of UV-absorbent Formulations

| Composition | Suitable Range | Preferred Range |
|---|---|---|
| Meⁿ⁺ UV-absorbent | 0.1-70% | 1-12% |

Suitable ratios of one or more of the following components

| | | |
|---|---|---|
| Cya Meⁿ⁺ | 0.01-20% | 10-15% |
| Pet Meⁿ⁺ | 0.01-20% | 1-10% |
| PolyAcyl Meⁿ⁺ | 0.1-20% | 0.1-15% |
| Stabilizer | 0.1-30% | 0.1-25% |
| Citric acid | 0.1-25% | 5-20% |
| Ascorbic acid | 0.1-25% | 0.1-5% |
| Excipients, Inducers, Meⁿ⁺ | (Optional) | |
| Dipotassium phosphate | 1-10% | 1-3% |
| Silicon dioxide | 1-10% | 1-3% |
| Flavor | 10-60% | 15-25% |
| Inducer (Optional) | | |
| Flavins | 2-20 ppm | 2-5 ppm |
| NH₄HCO₃ | 0-0.1% | 0.002-0.010% |
| Rebaudiosides | 0-3% | 0.05-2% |
| Dextrose | 0-50% | 10-50% |

Meⁿ⁺ascorbates, Meⁿ⁺citrates, Meⁿ⁺fumarates (Optional)

| | | |
|---|---|---|
| Fe | 0-45 ppm | 0.1-1 ppm |
| Mn | 0-100 ppb | 5-50 ppb |
| Zn | 0-20 ppm | 0.1-1 ppm |

Daily doses are administered orally 1 to 5-times per day, each 5-30 gm formula dissolved in 0.1-2.5 L water.

Exemplary Power Antioxidant CyaCFS
Meⁿ⁺ Starter and Maintenance Package

| Starter Dose Composition | Suitable Range | | Preferred Range | |
|---|---|---|---|---|
| Meⁿ⁺ UV-absorbent | | | | |
| CyaCFS Meⁿ⁺ | 3-12 | gm | 3-9 | gm |

| | | | |
|---|---|---|---|
| Stabilizer | Citric Acid | 0.1-25% | 1-5% |
| | Ascorbic acid | 0.1-25% | 0.3-1% |

Meⁿ⁺ ascorbates and/or Meⁿ⁺ fumarates (Optional)

| | | | | |
|---|---|---|---|---|
| Fe | 0.01-45 | ppm | 0.1-1 | ppm |
| Mn | 1-100 | ppb | 5-50 | ppb |
| Zn | 0.1-20 | ppm | 0.1-1 | ppm |

For a quick start, 6 to 24 hours before exposure, stir one Starter serving into 3-12 fl. oz. juice, tea, liquor, and/or water; and drink entire contents the day prior to exposure, preferably at least 15 minutes before breakfast.

| Maintenance Dose Composition | Suitable Range | | Preferred Range | |
|---|---|---|---|---|
| Meⁿ⁺ UV-absorbent | | | | |
| CyaCFS Meⁿ⁺ | 0.5-12 | gm | 0.5-3 | gm |

| | | | |
|---|---|---|---|
| Stabilizer | Citric Acid | 0.1-25% | 5-20% |
| | Ascorbic acid | 0.1-25% | 0.1-5% |

Meⁿ⁺citrates and/or Meⁿ⁺ascorbates (Optional)

| | | | | |
|---|---|---|---|---|
| Fe | 0.01-45 | ppm | 0.1-1 | ppm |
| Mn | 1-100 | ppb | 5-50 | ppb |
| Zn | 0.1-20 | ppm | 0.1-1 | ppm |

A single packet of dry powder is dissolved in 0.1-2.5 L water. Daily maintenance within the suitable range is administered orally.

EXAMPLE 4
Methods and Formulations for a Carbonated Beverage

| Composition | Suitable Range | Preferred Range |
|---|---|---|
| Meⁿ⁺ UV-absorbent | 0.1-70% | 1-12% |

Suitable ratios of one or more of the following components

| | | |
|---|---|---|
| Cya | 0.1-90% | 1-80% |
| Del | 0.1-90% | 1-80% |
| Pet | 0.1-90% | 1-80% |
| PolyAcyl | 0.1-10% | 0.1-5% |
| Stabilizer | 0.1-30% | 0.1-25% |
| Phosphoric Acid | 0.025-0.085% | 0.05-0.07% |
| Carbonic Acid | 0.1-1% | 0.2-0.5% |
| | | pH 2-4.2 |

### EXAMPLE 5 Exemplary Meⁿ⁺ UV-absorbent Mineral Supplements

Mineral supplement formulations that also provide systemic protection against damaging effects of UV radiation to the skin of an animal comprise one or more Meⁿ⁺ UV-absorbent in the form of a 2-(3,4-dihydroxyphenyl)chromenylium-3,5,7-triol 3-xylosyl(acylglucosyl)galactoside Meⁿ⁺, preferably in a CyaCFS Meⁿ⁺ blend. The preferred CyaCFS Fe exhibited higher incident sunlight absorbance than the CyaCFS Control solution, as shown in FIG. 6, and furthermore is a mineral supplement useful for the treatment or prevention of low blood levels of iron, such as caused by anemia and malnutrition. Specifically, FIG. 6 shows a shift to absorbance of sunlight irradiation after formation of the CyaCFS Fe coordination complex (CyaCFS+iron(II), right) that is darker, as compared to the CyaCFS Control solution (left) that is lighter, exhibiting less absorbance. “CyaCFS” refers to a blend of Meⁿ⁺ UV-absorbents selected from one or more of 2-(3,4-dihydroxyphenyl)chromenylium-3,5,7-triol 3-xylosyl(caffeoylglucosyl)galactosides; 2-(3,4-dihydroxyphenyl)chromenylium-3,5,7-triol 3-xylosyl(coumaroylglucosyl)galactosides; 2-(3,4-dihydroxyphenyl)chromenylium-3,5,7-triol 3-xylosyl(feruloylglucosyl)galactosides; 2-(3,4-dihydroxyphenyl)chromenylium-3,5,7-triol 3-xylosyl(sinapoylglucosyl)galactosides; and “CyaCFS Fe” refers to the Meⁿ⁺ UV-absorbent with iron(II) mineral supplement. Below, “CyaCFS Zn” refers to the Meⁿ⁺ UV-absorbent with zinc(II) mineral supplement.

| | Contents | | Range Preferred | |
|---|---|---|---|---|
| Iron Supplement | | | | |
| Meⁿ⁺ UV-absorbent | | | | |
| CyaCFS Fe | 300-3000 | mg | 500-2000 | mg |
| Stabilizer | | | | |
| Ascorbic acid | 10-1000 | mg | 10-50 | mg |
| Fe fumarate | 1-50 | mg | 10-45 | mg |
| Zinc Supplement | | | | |
| Meⁿ⁺ UV-absorbent | | | | |
| CyaCFS Zn | 300-3000 | mg | 500-2000 | mg |
| Stabilizer | | | | |
| Ascorbic acid | 10-1000 | mg | 10-50 | mg |
| Zn ascorbates | 1-40 | mg | 2-15 | mg |
| Alternative Components | | | | |
| Zn [as zinc citrate] | 1-50 | mg | 3-15 | mg |
| Mn [as manganese ascorbate] | 0.2-2 | mg | 0.5-1 | mg |
| Mo [as molybdenum glycinate chelate] | 2.5-250 | mcg | 2.5-25 | mcg |
| V [as vanadyl sulfate] | 2.5-750 | mcg | 2.5-25 | mcg |
| Fe [as ferrous fumarate] | 0.001-50 | mg | 1-45 | mg |
| 2-(3,4,5-trihydroxyphenyl)chromenylium- | 300-3000 | mg. | 500-2000 | mg |
| 3,5,7-triol 3-glycosyl(acylglucosyl)glycosides | | | | |
| 2-(3,4-dihydroxy-5-methoxyphenyl)chromenylium- | 300-3000 | mg | 500-2000 | mg |
| 3,5,7-triol3-glycosyl(acylglucosyl)glycosides | | | | |
| CyaCFS Mg | 300-3000 | mg | 500-2000 | mg |
| CyaCFS Mn | 300-3000 | mg | 500-2000 | mg |
| Excipients and Inducers (Optional) | | | | |
| Microcrystalline cellulose | | | | |
| Vegetable cellulose (capsule) | | | | |
| Maltodextrin | | | | |
| Starch | | | | |
| Stearic acid | | | | |
| UV-absorbent | | | | |
| Flavin | | | | |

| | |
|---|---|
| Malic acid | pH 2-4.1 |

Dosage and use: Take one or more 1000 mg capsules daily with food, preferably 1-3 gm Meⁿ⁺
UV-absorbent mineral supplement, or as recommended by a healthcare practitioner.

Exemplary Encapsulated CyaCFS Fe and CyaCFS
Zn Mineral Supplement for Sun Protection

| | Range, | | Preferred, | |
|---|---|---|---|---|
| Contents | mg/Gelcap | | mg/Gelcap | |
| Meⁿ⁺ UV-Absorbent | 300-3000 | mg | 500-2000 | mg |
| Stabilizer | | | | |
| Ascorbic acid | 10-1000 | mg | 10-50 | mg |
| Zinc ascorbate hydrate | 0.01-50 | mg | 2-10 | mg |
| Ferrous gluconate | 0.1-250 | mg | 0.2-5 | mg |
| Ingredients | | | | |

CyaCFS as Exberry ® Burgundy; Vitamin C as ascorbic acid and Zn ascorbate;
iron(II) as Fe gluconate
Dosage and use: Take one or more 1000 mg capsules daily, or as recommended by a healthcare
practitioner. The preferred orally administered recommended dose is in the range
of 1 to 3 gm/day.

1. A method for the systemic protection of an animal from exposure to a photocarcinogen, comprising orally administering to said animal a therapeutically effective amount of a formulation comprising one or more metalⁿ⁺ UV-absorbents comprising one or more 2-phenylchromenylium-acylₐglycoside_{g} metalⁿ⁺ and one or more stabilizers present in the formulation in an amount effective to achieve a pH of from 1-4.5 of the formulation; and wherein a and g each independently represents an integer from 1-8 and n in n+ represents an integer from 2-4.

2. The method of claim 1, wherein a represents an integer from 1-5.

3. The method of claim 1, wherein said animal is a human.

4. The method of claim 1, wherein said oral administration comprises a starter dose taken 6-24 hours prior to said exposure to a photocarcinogen, in the range of 3 to 12 grams metalⁿ⁺ UV-absorbent; and a maintenance dose taken the day following said administration of said starter dose, said maintenance dose being in the range 0.5 to 3 grams metalⁿ⁺ UV-absorbent; and where n in n+ represents an integer from 2-4.

5. The method of claim 1, wherein said therapeutically effective amount of said formulation comprises 0.5 to 3 grams of said one or more metalⁿ⁺ UV-absorbents administered per day, taken for 3 to 7 days prior to said exposure to a photocarcinogen, followed by 0.5 to 3 grams metalⁿ⁺ UV-absorbent administered per day for the duration of said exposure of said animal to a photocarcinogen; and where n in n+ represents an integer from 2-4.

6. The method of claim 1, wherein said metalⁿ⁺ UV-absorbent is selected from the group consisting of: (i) 3′,4′-dihydroxy-chromenylium-copigment selected from 2-(3,4-dihydroxyphenyl)chromenylium-3,5,7-triol, 2-(3,4-dihydroxy-5-methoxyphenyl)chromenylium-3,5,7-triol, and (2-(3,4,5-trihydroxyphenyl)chromenylium-3,5,7-triol; (ii) Acyl-copigment selected from the group consisting of aliphatic acids, acetic, malic, malonic, oxalic, succinic, tartaric acids; aromatic acids, caffeic, coumaric, ferulic, gallic, hydroxybenzoic, and sinapic acids; acyl, oligoacyl, polyacyl, thereof; (iii) Glycosyl-copigment selected from the group consisting of hexoses, glucose, rhamnose, galactose, pentoses, xylose, arabinose, rutinose, sophorose, samubiose; glycosides, oligoglycosyl, and polyglycosyl derivatives thereof; and (iv) Metalⁿ⁺ copigment selected from the group consisting of Fe, Mg, Mn, V, Zn and combinations thereof; and combinations of (i), (ii), (iii) and/or (iv).

7. The method of claim 1, wherein said metalⁿ⁺ UV-absorbent is selected from the group consisting of 2-(3,4-dihydroxyphenyl) chromenylium-3,5,7-triol 3-xylosyl (acylglucosyl) galactosides metalⁿ⁺; 2-(3,4-dihydroxyphenyl) chromenylium-3,5,7-triol 3-xylosyl (feruloylglucosyl) galactosides metalⁿ⁺; 2-(3,4-dihydroxyphenyl) chromenylium-3,5,7-triol 3-xylosyl (sinapoylglucosyl) galactosides metalⁿ⁺; 2-(3,4-dihydroxyphenyl) chromenylium-3,5,7-triol 3-xylosyl (coumaroylglucosyl) galactosides metalⁿ⁺; 2-(3,4-dihydroxyphenyl) chromenylium-3,5,7-triol 3-xylosyl (caffeoylglucosyl) galactoside metalⁿ⁺; and combinations thereof, in the presence of a metalⁿ⁺ selected from the group consisting of Fe, Mg, Mn, V, and Zn and combinations thereof; and where n in n+ represents an integer from 2-4.

8. The method of claim 1, wherein a source of said one or more metalⁿ⁺ UV-absorbents comprises an extract from purple carrot, Daucus carota sativus var. atrorubens; in the presence of a metalⁿ⁺ copigment selected from the group consisting of Fe, Mg, Mn, V, and Zn and combinations thereof.

9. The method of claim 1, wherein said metalⁿ⁺ UV-absorbent is selected from the group consisting of 2-(3,4-dihydroxyphenyl) chromenylium-3,5,7-triol 3-acylₐglycoside_{g} metalⁿ⁺; 2-(3,4-dihydroxy-5-methoxyphenyl) chromenylium-3,5,7-triol 3-acylₐglycoside_{g} metalⁿ⁺; 2-(3,4,5-trihydroxyphenyl) chromenylium-3,5,7-triol 3-acylₐglycoside_{g} metalⁿ⁺; 2-(3,4-dihydroxyphenyl) chromenylium-3,5,7-triol 3-(coumaroylsophoroside)-5-glucoside metalⁿ⁺; 2-(3,4-dihydroxyphenyl) chromenylium-3,5,7-triol 3-(coumaroyldiglucosyl)-5-glucoside metalⁿ⁺; 2-(3,4-dihydroxyphenyl) chromenylium-3,5,7-triol 3-(sinapoyldiglucosyl)-5-glucoside metalⁿ⁺; 2-(3,4-dihydroxyphenyl) chromenylium-3,5,7-triol 3-(dicaffeoylsophoroside)-5-glucoside metalⁿ⁺; 2-(3,4-dihydroxyphenyl)chromenylium-3,5,7-triol 3-(caffeoyl-6-feruloylsophoroside)-5-glucoside metalⁿ⁺; 2-(3,4-dihydroxyphenyl) chromenylium-3,5,7-triol 3-(p-hydroxybenzoyl-caffeoylsophoroside)-5-glucoside metalⁿ⁺; 2-(3,4-dihydroxyphenyl) chromenylium-3,5,7-triol 3-(coumaroylsophorosyl)-5-sinapoylglucoside metalⁿ⁺; 2-(3,4-dihydroxyphenyl) chromenylium-3,5,7-triol 3-xylosyl (sinapoylglucosyl) galactoside metalⁿ⁺; 2-(3,4-dihydroxyphenyl) chromenylium-3,5,7-triol 3-xylosyl (feruloylglucosyl) galactosides metalⁿ⁺; 2-(3,4-dihydroxyphenyl)chromenylium-3,5,7-triol 3-xylosyl(coumaroylglucosyl)galactosides metalⁿ⁺; 2-(3,4-dihydroxyphenyl)chromenylium-3,5,7-triol 3-xylosyl(caffeoylglucosyl)galactosides metalⁿ⁺; 2-(3,4-dihydroxy-5-methoxyphenyl)chromenylium-3,5,7-triol 3-(p-coumaroylrutinoside)-5-glucoside metalⁿ⁺; 2-(3,4,5-trihydroxyphenyl)chromenylium-3,5,7-triol 3-(p-coumaroylrutinoside)-5-glucoside metalⁿ⁺; 2-(3,4-dihydroxyphenyl)chromenylium-3,5,7-triol 3-(maloylsuccinyl)glucosides metalⁿ⁺; 2-(3,4-dihydroxyphenyl)chromenylium-3,5,7-triol 3-malonylglucoside metalⁿ⁺; 2-(3,4-dihydroxyphenyl)chromenylium-3,5,7-triol 3-(dimalonylglycosyl)glucosides metalⁿ⁺; 2-(3,4-dihydroxyphenyl)chromenylium-3,5,7-triol 3-(coumaroylsophorosyl)-5-malonylglucosides metalⁿ⁺; 2-(3,4-dihydroxyphenyl)chromenylium-3,5,7-triol 3-(feruloylsophorosyl)-5-malonylglucosides metalⁿ⁺; 2-(3,4-dihydroxyphenyl)chromenylium-3,5,7-triol 3-(sinapoyl-coumaroyl)sophorosyl-5-malonylglucosides metalⁿ⁺; 2-(3,4-dihydroxyphenyl)chromenylium-3,5,7-triol 3-(sinapoyl-feruloyl)sophorosyl-5-malonylglucosides metalⁿ⁺; 2-(3,4-dihydroxyphenyl)chromenylium-3,5,7-triol 3-(feruloylglucosyl)-6-(glucosylferuloyl) (glucosylcoumaroyl)glucoside)-5-6-(malonylglucoside) metalⁿ⁺, and combinations thereof; in the presence of one or more metalⁿ⁺ copigments selected from the group consisting of Fe, Mg, Mn, V and Zn, where n in n+ represents an integer from 2-4; and where a represents an integer from 1-5, and g represents an integer from 1 to 8.

10. The method of claim 1, wherein said one or more stabilizers is selected from the group consisting of acetic acid, ascorbic acid, carbonic acid, citric acid, fumaric acid, gluconic acid, glutaric acid, lactic acid, malic acid, phosphoric acid, succinic acid, tartaric acid and metalⁿ⁺ salts thereof; and where n in n+ represents an integer from 2-4.

11. The method of claim 1, wherein said photocarcinogen is one or more of sunlight, UVA and UVB.

12. The method of claim 1, wherein said administration improves the appearance of skin of said animal.

13. The method of claim 12, wherein said improvement of the appearance of skin comprises the mitigation of at least one of skin wrinkling, skin sagging, skin thickening, and fine lines.

14. The method of claim 1, wherein said administration mitigates or prevents photowrinkling of skin of said animal.

15. A formulation for the systemic protection against damaging effects of UV radiation to the skin of an animal, comprising one or more metalⁿ⁺ UV-absorbents comprising a 2-phenylchromenylium-acylₐglycoside_{g} metalⁿ⁺ copigment in an amount of 0.1%-70% of the formulation, wherein a and g each independently represent an integer from 1-8 and n in n+ represents an integer from 2-4; and one or more stabilizers in an amount of 0.1-30% of the formulation so as to achieve a pH of said formulation in the range of 1-4.5.

16. The formulation of claim 15, wherein said metalⁿ⁺ UV-absorbent is selected from the group consisting of: (i) a 3′,4′-dihydroxy-chromenylium copigment selected from 2-(3,4-dihydroxyphenyl)chromenylium-3,5,7-triol and 2-(3,4-dihydroxy-5-methoxyphenyl) chromenylium-3,5,7-triol; 2-(3,4,5-trihydroxyphenyl)chromenylium-3,5,7-triol; (ii) Acyl-copigment comprising one or more of aliphatic acids, acetic, malic, malonic, oxalic, succinic, tartaric acids; aromatic acids, caffeic, coumaric, ferulic, gallic, hydroxybenzoic, and sinapic acids; and acyl, oligoacyl and polyacyl derivatives thereof; (iii) Glycosyl-copigment comprising one or more of hexoses, glucose, rhamnose, galactose, pentoses, xylose, arabinose, rutinose, sophorose, samubiose; glycoside, oligoglycosyl and polyglycosyl derivatives thereof; (iv) Metalⁿ⁺ copigment comprising one or more of Fe, Mg, Mn, V, and Zn; combinations of (i), (ii), (iii) and (iv); where n in n+ represents an integer from 2-4; and derivatives thereof.

17. The formulation of claim 15, wherein said metalⁿ⁺ UV-absorbent comprises a 3′,4′-dihydroxy-chromenylium copigment selected from the group consisting of 2-phenyl-3′,4′-dihydroxy-chromenylium-acylₐglycoside_{g} metalⁿ⁺; 2-(3,4-dihydroxyphenyl)chromenylium-3,5,7-triol 3-acylₐglycoside_{g} metalⁿ⁺, 2-(3,4,5-trihydroxyphenyl)chromenylium-3,5,7-triol 3-acylₐglycoside_{g} metalⁿ⁺; 2-(3,4-dihydroxy-5-methoxyphenyl)chromenylium-3,5,7-triol 3-acylₐglycoside_{g} metalⁿ⁺; 2-(3,4-dihydroxyphenyl)chromenylium-3,5,7-triol 3-xylosyl (sinapoylglucosyl)galactoside metalⁿ⁺, 2-(3,4-dihydroxyphenyl)chromenylium-3,5,7-triol 3-xylosyl(feruloylglucosyl)galactosides metalⁿ⁺, 2-(3,4-dihydroxyphenyl)chromenylium-3,5,7-triol 3-xylosyl(coumaroylglucosyl)galactosides metalⁿ⁺, 2-(3,4-dihydroxyphenyl)chromenylium-3,5,7-triol 3-xylosyl (caffeoylglucosyl)galactosides metalⁿ⁺; 2-(3,4-dihydroxyphenyl)chromenylium-3,5,7-triol 3-(coumaroylsophoroside)-5-glucoside metalⁿ⁺; 2-(3,4-dihydroxyphenyl)chromenylium-3,5,7-triol 3-(coumaroyldiglucosyl)-5-glucoside metalⁿ⁺; 2-(3,4-dihydroxyphenyl)chromenylium-3,5,7-triol 3-(sinapoyldiglucosyl)-5-glucoside metalⁿ⁺; 2-(3,4-dihydroxyphenyl)chromenylium-3,5,7-triol 3-(dicaffeoylsophoroside)-5-glucoside metalⁿ⁺; 2-(3,4-dihydroxyphenyl)chromenylium-3,5,7-triol 3-(caffeoyl-6-feruloylsophoroside)-5-glucoside metalⁿ⁺; 2-(3,4-dihydroxyphenyl)chromenylium-3,5,7-triol 3-(p-hydroxybenzoyl-caffeoylsophoroside)-5-glucoside metalⁿ⁺; 2-(3,4-dihydroxyphenyl)chromenylium-3,5,7-triol 3-(coumaroylsophorosyl)-5-sinapoylglucoside metalⁿ⁺; 2-(3,4,5-trihydroxyphenyl)chromenylium-3,5,7-triol 3-(p-coumaroylrutinoside)-5-glucoside metalⁿ⁺; 2-(3,4-dihydroxy-5-methoxyphenyl)chromenylium-3,5,7-triol 3-(p-coumaroylrutinoside)-5-glucoside metalⁿ⁺; 2-(3,4-dihydroxyphenyl)chromenylium-3,5,7-triol 3-malonylglucoside metalⁿ⁺; 2-(3,4-dihydroxyphenyl)chromenylium-3,5,7-triol 3-(dimalonylglycosyl)glucosides metalⁿ⁺; 2-(3,4-dihydroxyphenyl)chromenylium-3,5,7-triol 3-(maloylsuccinyl)glucosides metalⁿ⁺; 2-(3,4-dihydroxyphenyl)chromenylium-3,5,7-triol 3-(coumaroylsophorosyl)-5-malonylglucosides metalⁿ⁺; 2-(3,4-dihydroxyphenyl)chromenylium-3,5,7-triol 3-(feruloylsophorosyl)-5-malonylglucosides metalⁿ⁺; 2-(3,4-dihydroxyphenyl)chromenylium-3,5,7-triol 3-(sinapoyl-coumaroyl)sophorosyl-5-malonylglucosides metalⁿ⁺; 2-(3,4-dihydroxyphenyl)chromenylium-3,5,7-triol 3-(sinapoyl-feruloyl) sophorosyl-5-malonylglucosides metalⁿ⁺; and 2-(3,4-dihydroxyphenyl) chromenylium-3,5,7-triol 3-(feruloylglucosyl)-6-(glucosylferuloyl) (glucosylcoumaroyl) glucoside)-5-6-(malonylglucoside) metalⁿ⁺; in the presence of metalⁿ⁺ copigments selected from the group consisting of Fe, Mg, Mn, Zn and combinations thereof, where n in n+ represents an integer from 2-4; a represents an integer from 1-5, g represents an integer from 1-8, and n in n+ represents an integer from 2-4.

18. The formulation of claim 15, further comprising one or more inducers comprising a riboflavin Na phosphate; said inducer further comprising one or more ammonium compounds selected from the group consisting of ammonium sulfate, NH₄HCO₃, and C₄₂H₆₅NO₁₆; said inducer being present in said formulation in the amount from 1 to 100 ppm.

19. The formulation of claim 15, wherein said one or more metalⁿ⁺ UV-absorbents is selected from the group consisting of 2-(3,4-dihydroxyphenyl)chromenylium-3,5,7-triol 3-xylosyl(acylglucosyl)galactosides Fe; 2-(3,4-dihydroxyphenyl) chromenylium-3,5,7-triol 3-xylosyl(feruloylglucosyl)galactosides Fe; 2-(3,4-dihydroxyphenyl) chromenylium-3,5,7-triol 3-xylosyl(sinapoylglucosyl)galactosides Fe; 2-(3,4-dihydroxyphenyl)chromenylium-3,5,7-triol 3-xylosyl(coumaroylglucosyl)galactosides Fe; 2-(3,4-dihydroxyphenyl)chromenylium-3,5,7-triol 3-xylosyl(caffeoylglucosyl)galactoside Fe; 2-(3,4-dihydroxyphenyl)chromenylium-3,5,7-triol 3-xylosyl(acylglucosyl)galactosides Zn; 2-(3,4-dihydroxyphenyl)chromenylium-3,5,7-triol 3-xylosyl(feruloylglucosyl)galactosides Zn; 2-(3,4-dihydroxyphenyl)chromenylium-3,5,7-triol 3-xylosyl(sinapoylglucosyl)galactosides Zn; 2-(3,4-dihydroxyphenyl)chromenylium-3,5,7-triol 3-xylosyl(coumaroylglucosyl)galactosides Zn; and 2-(3,4-dihydroxyphenyl)chromenylium-3,5,7-triol 3-xylosyl(caffeoylglucosyl)galactoside Zn; in the presence of one or more metalⁿ⁺ copigments selected from Fe and Zn, wherein n in n+ represents an integer from 2-4; and mineral supplements thereof.

20. The formulation of claim 15 contained in a housing impervious to light.

21. The formulation of claim 15, wherein said one or more metalⁿ⁺ UV-absorbents is selected from the group consisting of 2-(3,4-dihydroxyphenyl)chromenylium-3,5,7-triol 3-acylₐglycoside_{g} metalⁿ⁺ in an amount of 0.5-12 grams dry weight; and wherein said formulation comprises ascorbic acid in the amount of 10-1000 mg dry weight; flavins in the amount of 1-100 ppm dry weight; ammonium bicarbonate in the amount of 1-100 ppm dry weight; zinc ascorbate in the amount of 0.01-50 mg dry weight; and ferrous fumarate in the amount of 1 mcg-50 mg dry weight; wherein said formulation is for a human dose; where a represents an integer from 1-5, g represents an integer from 1 to 8, and n in n+ represents an integer from 2-4.

22. A formulation for the systemic protection against damaging effects of UV radiation to skin of an animal, comprising one or more metalⁿ⁺ UV-absorbents selected from the group consisting of 2-phenylchromenylium-acylₐglycoside_{g} metalⁿ⁺; 2-(3,4-dihydroxyphenyl)chromenylium-3,5,7-triol 3-acylₐglycoside_{g} metalⁿ⁺; 2-(3,4-dihydroxyphenyl) chromenylium-3,5,7-triol 3-xylosyl(sinapoylglucosyl)galactoside metalⁿ⁺, 2-(3,4-dihydroxyphenyl)chromenylium-3,5,7-triol 3-xylosyl(feruloylglucosyl)galactosides metalⁿ⁺, 2-(3,4-dihydroxyphenyl)chromenylium-3,5,7-triol 3-xylosyl(coumaroylglucosyl)galactosides metalⁿ⁺, 2-(3,4-dihydroxyphenyl)chromenylium-3,5,7-triol 3-xylosyl (caffeoylglucosyl)galactosides metalⁿ⁺; 2-(3,4,5-trihydroxyphenyl)chromenylium-3,5,7-triol 3-acylₐglycoside_{g} metalⁿ⁺; 2-(3,4-dihydroxy-5-methoxyphenyl)chromenylium-3,5,7-triol 3-acylₐglycoside_{g} metalⁿ⁺; 2-(3,4-dihydroxyphenyl)chromenylium-3,5,7-triol 3-(coumaroylsophoroside)-5-glucoside metalⁿ⁺; 2-(3,4-dihydroxyphenyl)chromenylium-3,5,7-triol 3-(coumaroyldiglucosyl)-5-glucoside metalⁿ⁺; 2-(3,4-dihydroxyphenyl)chromenylium-3,5,7-triol 3-(sinapoyldiglucosyl)-5-glucoside metalⁿ⁺; 2-(3,4-dihydroxyphenyl)chromenylium-3,5,7-triol 3-(dicaffeoylsophoroside)-5-glucoside metalⁿ⁺; 2-(3,4-dihydroxyphenyl)chromenylium-3,5,7-triol 3-(caffeoyl-6-feruloylsophoroside)-5-glucoside metalⁿ⁺; 2-(3,4-dihydroxyphenyl)chromenylium-3,5,7-triol 3-(p-hydroxybenzoyl-caffeoylsophoroside)-5-glucoside metalⁿ⁺; 2-(3,4-dihydroxyphenyl)chromenylium-3,5,7-triol 3-(coumaroylsophorosyl)-5-sinapoylglucoside metalⁿ⁺; 2-(3,4,5-trihydroxyphenyl)chromenylium-3,5,7-triol 3-(p-coumaroylrutinoside)-5-glucoside metalⁿ⁺; 2-(3,4-dihydroxy-5-methoxyphenyl)chromenylium-3,5,7-triol 3-(p-coumaroylrutinoside)-5-glucoside metalⁿ⁺; 2-(3,4-dihydroxyphenyl)chromenylium-3,5,7-triol 3-malonylglucoside metalⁿ⁺; 2-(3,4-dihydroxyphenyl)chromenylium-3,5,7-triol 3-(dimalonylglycosyl)glucosides metalⁿ⁺; 2-(3,4-dihydroxyphenyl)chromenylium-3,5,7-triol 3-(maloylsuccinyl)glucosides metalⁿ⁺; 2-(3,4-dihydroxyphenyl)chromenylium-3,5,7-triol 3-(coumaroylsophorosyl)-5-malonylglucosides metalⁿ⁺; 2-(3,4-dihydroxyphenyl) chromenylium-3,5,7-triol 3-(feruloylsophorosyl)-5-malonylglucosides metalⁿ⁺; 2-(3,4-dihydroxyphenyl) chromenylium-3,5,7-triol 3-(sinapoyl-coumaroyl) sophorosyl-5-malonylglucosides metalⁿ⁺; and 2-(3,4-dihydroxyphenyl) chromenylium-3,5,7-triol 3-(sinapoyl-feruloyl) sophorosyl-5-malonylglucosides metalⁿ⁺; and 2-(3,4-dihydroxyphenyl) chromenylium-3,5,7-triol 3-(feruloylglucosyl)-6-(glucosylferuloyl) (glucosylcoumaroyl) glucoside)-5-6-(malonylglucoside) metalⁿ⁺; in the presence of one or more metalⁿ⁺ copigments selected from the group consisting of Fe, Mg, Mn, V and Zn, where n in n+ represents an integer from 2-4; a represents an integer from 1-5 and g represents an integer from 1-8; and combinations thereof.
